SEQUENCE LISTING (13 sequences)

SEQ ID NO: 1 - DNA, 1071 bp, Homo sapiens
  source 1..1071: mol_type = genomic DNA
atggatgccggtgtgactgaaagtggactaaatgtgactctcaccattcggcttcttatgcacggaaaggaagtaggaagcatcattgggaagaaaggggagtcggttaagaggatccgcgaggagagtggcgcgcggatcaacatctcggaggggaattgtccggagagaatcatcactctgaccggccccaccaatgccatctttaaggctttcgctatgatcatcgacaagctggaggaagatatcaacagctccatgaccaacagtaccgcggccagcaggcccccggtcaccctgaggctggtggtgccggccacccagtgcggctccctgattgggaaaggcgggtgtaagatcaaagagatccgcgagagtacgggggcgcaggtccaggtggcgggggatatgctgcccaactccaccgagcgggccatcaccatcgctggcgtgccgcagtctgtcaccgagtgtgtcaagcagatttgcctggtcatgctggagacgctctcccagtctccgcaagggagagtcatgaccattccgtaccagcccatgccggccagctccccagtcatctgcgcgggcggccaagatcggtgcagcgacgctgcgggctacccccatgccacccatgacctggagggaccacctctagatgcctactcgattcaaggacaacacaccatttctccgctcgatctggccaagctgaaccaggtggcaagacaacagtctcactttgccatgatgcacggcgggaccggattcgccggaattgactccagctctccagaggtgaaaggctattgggcaagtttggatgcatctactcaaaccacccatgaactcaccattccaaataacttaattggctgcataatcgggcgccaaggcgccaacattaatgagatccgccagatgtccggggcccagatcaaaattgccaacccagtggaaggctcctctggtaggcaggttactatcactggctctgctgccagtattagtctggcccagtatctaatcaatgccaggctttcctctgagaagggcatggggtgcagctag

SEQ ID NO: 2 - protein, 356 aa, Homo sapiens
MDAGVTESGLNVTLTIRLLMHGKEVGSIIGKKGESVKRIREESGARINISEGNCPERIITLTGPTNAIFKAFAMIIDKLEEDINSSMTNSTAASRPPVTLRLVVPATQCGSLIGKGGCKIKEIRESTGAQVQVAGDMLPNSTERAITIAGVPQSVTECVKQICLVMLETLSQSPQGRVMTIPYQPMPASSPVICAGGQDRCSDAAGYPHATHDLEGPPLDAYSIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 3 - DNA, 1071 bp, synthetic construct
  misc_feature 1..1071: note = mPCBP1_1 (c668t)
  source 1..1071: mol_type = other DNA
atggatgccggtgtgactgaaagtggactaaatgtgactctcaccattcggcttcttatgcacggaaaggaagtaggaagcatcattgggaagaaaggggagtcggttaagaggatccgcgaggagagtggcgcgcggatcaacatctcggaggggaattgtccggagagaatcatcactctgaccggccccaccaatgccatctttaaggctttcgctatgatcatcgacaagctggaggaagatatcaacagctccatgaccaacagtaccgcggccagcaggcccccggtcaccctgaggctggtggtgccggccacccagtgcggctccctgattgggaaaggcgggtgtaagatcaaagagatccgcgagagtacgggggcgcaggtccaggtggcgggggatatgctgcccaactccaccgagcgggccatcaccatcgctggcgtgccgcagtctgtcaccgagtgtgtcaagcagatttgcctggtcatgctggagacgctctcccagtctccgcaagggagagtcatgaccattccgtaccagcccatgccggccagctccccagtcatctgcgcgggcggccaagatcggtgcagcgacgctgcgggctacccccatgccacccatgacctggagggaccacctctagatgcctacttgattcaaggacaacacaccatttctccgctcgatctggccaagctgaaccaggtggcaagacaacagtctcactttgccatgatgcacggcgggaccggattcgccggaattgactccagctctccagaggtgaaaggctattgggcaagtttggatgcatctactcaaaccacccatgaactcaccattccaaataacttaattggctgcataatcgggcgccaaggcgccaacattaatgagatccgccagatgtccggggcccagatcaaaattgccaacccagtggaaggctcctctggtaggcaggttactatcactggctctgctgccagtattagtctggcccagtatctaatcaatgccaggctttcctctgagaagggcatggggtgcagctag

SEQ ID NO: 4 - protein, 356 aa, synthetic construct
  REGION 1..356: note = mPCBP1_1 (S223L)
MDAGVTESGLNVTLTIRLLMHGKEVGSIIGKKGESVKRIREESGARINISEGNCPERIITLTGPTNAIFKAFAMIIDKLEEDINSSMTNSTAASRPPVTLRLVVPATQCGSLIGKGGCKIKEIRESTGAQVQVAGDMLPNSTERAITIAGVPQSVTECVKQICLVMLETLSQSPQGRVMTIPYQPMPASSPVICAGGQDRCSDAAGYPHATHDLEGPPLDAYLIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 5 - DNA, 1071 bp, synthetic construct
  misc_feature 1..1071: note = mPCBP1_2 cDNA (a178g and a379g)
  source 1..1071: mol_type = other DNA
atggatgccggtgtgactgaaagtggactaaatgtgactctcaccattcggcttcttatgcacggaaaggaagtaggaagcatcattgggaagaaaggggagtcggttaagaggatccgcgaggagagtggcgcgcggatcaacatctcggaggggaattgtccggagagaatcatcgctctgaccggccccaccaatgccatctttaaggctttcgctatgatcatcgacaagctggaggaagatatcaacagctccatgaccaacagtaccgcggccagcaggcccccggtcaccctgaggctggtggtgccggccacccagtgcggctccctgattgggaaaggcgggtgtaagatcaaagagatccgcgagagtgcgggggcgcaggtccaggtggcgggggatatgctgcccaactccaccgagcgggccatcaccatcgctggcgtgccgcagtctgtcaccgagtgtgtcaagcagatttgcctggtcatgctggagacgctctcccagtctccgcaagggagagtcatgaccattccgtaccagcccatgccggccagctccccagtcatctgcgcgggcggccaagatcggtgcagcgacgctgcgggctacccccatgccacccatgacctggagggaccacctctagatgcctactcgattcaaggacaacacaccatttctccgctcgatctggccaagctgaaccaggtggcaagacaacagtctcactttgccatgatgcacggcgggaccggattcgccggaattgactccagctctccagaggtgaaaggctattgggcaagtttggatgcatctactcaaaccacccatgaactcaccattccaaataacttaattggctgcataatcgggcgccaaggcgccaacattaatgagatccgccagatgtccggggcccagatcaaaattgccaacccagtggaaggctcctctggtaggcaggttactatcactggctctgctgccagtattagtctggcccagtatctaatcaatgccaggctttcctctgagaagggcatggggtgcagctag

SEQ ID NO: 6 - protein, 356 aa, synthetic construct
  REGION 1..356: note = mPCBP1_2 (T60A and T127A)
MDAGVTESGLNVTLTIRLLMHGKEVGSIIGKKGESVKRIREESGARINISEGNCPERIIALTGPTNAIFKAFAMIIDKLEEDINSSMTNSTAASRPPVTLRLVVPATQCGSLIGKGGCKIKEIRESAGAQVQVAGDMLPNSTERAITIAGVPQSVTECVKQICLVMLETLSQSPQGRVMTIPYQPMPASSPVICAGGQDRCSDAAGYPHATHDLEGPPLDAYLIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 7 - DNA, 63 bp, unidentified
  misc_feature 1..63: note = T2A sequence in AAV vector
  source 1..63: mol_type = other DNA
ggaagcggagagggcaggggaagtcttctaacatgcggggacgtggaggaaaatcccggcccc

SEQ ID NO: 8 - DNA, 15 bp, synthetic construct
  misc_feature 1..15: note = mutant PCBP1 mutation region sequence
  source 1..15: mol_type = other DNA
gcctacttgattcaa

SEQ ID NO: 9 - DNA, 12 bp, synthetic construct
  misc_feature 1..12: note = mutant PCBP1 mutation region sequence
  source 1..12: mol_type = other DNA
atcatcgctctg

SEQ ID NO: 10 - DNA, 15 bp, synthetic construct
  misc_feature 1..15: note = mutant PCBP1 mutation region sequence
  source 1..15: mol_type = other DNA
gagagtgcgggggcg

SEQ ID NO: 11 - protein, 5 aa, synthetic construct
  REGION 1..5: note = mutant PCBP1 mutation region sequence
AYLIQ

SEQ ID NO: 12 - protein, 5 aa, synthetic construct
  REGION 1..5: note = mutant PCBP1 mutation region sequence
IIALT

SEQ ID NO: 13 - protein, 5 aa, synthetic construct
  REGION 1..5: note = mutant PCBP1 mutation region sequence
ESAGA

(19) United States
(12) Patent Application Publication
(10) Pub. No.: US 2025/0057978 A1
(43) Pub. Date: Feb. 20, 2025

(54) USE OF PCBP1 TO TREAT HYPERPROLIFERATIVE DISEASE

(71) Applicant: Ibex Biosciences, Inc., Cumberland, MD (US)

(72) Inventors: Norman Zhennan LAI, N. Potomac, MD (US); Michael Joseph Karlin, Bethesda, MD (US); Alberto Murat Croci, Washington, DC (US); Vidal Felix De La Cruz, JR., Phoenixville, PA (US); Yuebin Tan, Cumberland, MD (US)

(21) Appl. No.: 18/626,391

(22) Filed: Apr. 4, 2024

Related U.S. Application Data
(63) Continuation of application No. 16/967,507, filed on Aug. 5, 2020, now Pat. No. 11,980,670.
(60) Provisional application No. 62/626,424, filed on Feb. 5, 2018.

(51) Int. Cl. A61K 48/00; A61P 35/04; C12N 15/86

(52) U.S. Cl. CPC A61K 48/005; A61P 35/04; C12N 15/86

(57) ABSTRACT

The present disclosure provides compositions and methods of using gene therapy to decrease cell migration and metastasis and reduce tumor mass or burden. The methods disclosed herein employ plasmids, vectors, and non-viral vectors that contain PCBP1 and/or PCBP1 mutants which inhibit the expression of cancer biomarkers and decrease cell migration or tumor size.

### CROSS REFERENCE TO RELATED APPLICATIONS

This application is a continuation of U.S. patent application Ser. No. 16/967,507, filed Aug. 5, 2020, which is a U.S. national stage application of International Patent Application No. PCT/US2019/016688, filed Feb. 5, 2019, which claims benefit of U.S. Provisional Application No. 62/626,424, filed Feb. 5, 2018, the contents of which are incorporated by reference in their entirety for all purposes.

### FIELD

The present disclosure relates to compositions and methods to inhibit cell migration and cancer metastasis, and to decrease tumor burden. Specifically, the present disclosure provides methods and compositions for the transduction of cancer cells to inhibit cancer cell migration and cancer development and/or metastasis. The compositions may be administered in vitro, in vivo, ex vivo, or in situ.

### DESCRIPTION OF THE TEXT FILE SUBMITTED ELECTRONICALLY

The contents of the electronic sequence listing (IBEX_003_02US_SeqList_ST26.xml; Size: 20,932 bytes; and Date of Creation: Mar. 29, 2024) are herein incorporated by reference in its entirety.

### BACKGROUND

Phosphatase of Regenerating Liver 3 (PRL-3) is an oncogenic factor, the activation of which is often associated with tumorigenesis and metastasis. Previous studies have shown poly(rC) binding protein (PCBP1) decreases the expression of PRL-3, and may be able to inhibit the development of oncogenesis.

To realize the promise of novel gene therapies for cancer, the cancer cells must be manipulated so that cancer development and expression of biomarkers associated with tumorigenesis and metastasis are decreased or prevented. This will prevent development of cancer cells and/or keep them from becoming metastatic, and minimize the invasion of tissues and organs throughout the body.

### SUMMARY OF THE INVENTION

The present disclosure provides methods of inhibiting tumor development, growth, cell migration, and/or metastasis by using gene therapy to transfect or/and transduce a cancer cell with a composition encoding a PCBP1 polypeptide, mutants, and/or variants thereof.

In some aspects the present disclosure provides methods of inhibiting, preventing, or decreasing cell migration comprising introducing a vector comprising a PCBP1 or a mutant PCBP1 nucleic acid sequence into a cell. In some embodiments, the cell migration is metastasis.

In some aspects, the present disclosure provides methods of treating cancer comprising introducing a vector comprising a PCBP1 or a mutant PCBP1 nucleic acid sequence into a cancer cell.

In some embodiments, the method is performed in vitro. In some embodiments, the method is performed ex vivo. In some embodiments, the method is performed in vivo.

In some embodiments, the cells are mammalian. In some embodiments, the mammalian cells are human. In some embodiments, the mammalian cells are organ cells, tissue cells, or blood cells.

In some embodiments, the mammalian cells are cancer cells. In some embodiments, the cancer cells are cells from melanoma, prostate, pancreatic, glioblastoma, retinoblastoma or a breast cancer.

In some embodiments, the mutant PCBP1 nucleic acid sequence encodes a polypeptide that cannot be phosphorylated or not fully phosphorylated compared to wild type PCBP1. In some embodiments, the mutant PCBP1 is not phosphorylated or not fully phosphorylated by p21-activating kinases (PAK).

In some embodiments, the mutant PCBP1 nucleic acid comprises one, two, three, four, or five or more mutations compared to wild type PCBP1 (SEQ ID NO: 1). In some embodiments, the mutant PCBP1 nucleic acid has at least 75% percent identity with wild type PCBP1 (SEQ ID NO: 1). In some embodiments, the mutant PCPB1 nucleic acid comprises of SEQ ID NO: 3. In some embodiments, the mutant PCBP1 nucleic acid comprises the sequence of SEQ ID NO: 5.

In some embodiments, the vector comprises a mutant PCBP1 nucleic acid sequence that encodes a mutant PCBP1 polypeptide. In some embodiments, the mutant PCBP1 polypeptide is not phosphorylated or not fully phosphorylated compared to wild type PCBP1. In some embodiments, the mutant PCBP1 polypeptide is not phosphorylated or is not fully phosphorylated by the P21-activated kinase-1 (PAK1). In some embodiments, the mutant PCBP1 inhibits or prevents phosphorylated PCBP1 production.

In some embodiments, the mutant PCBP1 polypeptide comprises one, two, three, four, five or more mutations compared to wild type PCBP1 (SEQ ID NO: 2). In some embodiments, the mutant PCBP1 polypeptide has at least 75% percent identity with wild type PCBP1 (SEQ ID NO: 2). In some embodiments, the mutant PCPB1 polypeptide comprises the sequence of SEQ ID NO: 4. In some embodiments, the mutant PCBP1 polypeptide comprises the sequence of SEQ ID NO: 6.

In some embodiments, the cell expresses multiple copies of a wild type or mutant PCBP1 nucleic acid sequence. In some embodiments, the cell expresses multiple copies of a wild type or mutant PCBP1 polypeptide.

### BRIEF DESCRIPTION OF THE FIGURES

FIG. 1A shows a representative lentiviral vector containing wild type or mutant PCBP1 sequences. In this vector, PCBP1 is driven by an EF1a promoter and followed by a secondary expression cassette containing an IRES-GFP reporter gene cassette.

FIG. 1B shows a representative AAV vector containing wild type or mutant PCBP1 sequences. In this vector, expression of the PCBP1 sequence is driven by an EF1a promoter and followed by a secondary expression cassette containing an IRES-GFP reporter gene cassette. This vector can contain a WPRE fragment inserted after the GFP sequence to enhance the expression of the transgene(s).

FIG. 2A shows qRT-PCR analysis of gene expression in melanoma cells transfected with the compositions of the instant disclosure which measures the fold change between groups (ddct): GFP-only vs GFP plus wild type PCBP1 (wPCBP1_0) shows upregulation of PCBP1 with expression 4.012 fold higher; GFP-only vs GFP plus PCBP1 S223L (mPCBP1_1) shows PCBP1 expression 3.97 higher; and GFP-only vs GFP plus PCBP1 T60A & T127A (mPCBP1_2) shows PCBP1 expression 4.02 fold higher; respectively (*p<0.01). The housekeeping gene is GAPDH.

FIG. 2B shows GFP expression in melanoma cells after in vitro transfection of the therapeutic gene vectors of this disclosure. (A): GFP only; (B) wPCBP1_0-IRES-GFP; (C) mPCBP1_1-IRES-GFP; (D): mPCBP1_2-IRES-GFP. (Scale bar: 200 μm).

FIG. 3 shows PRL-3 protein expression by Western Blot analysis after treatment by gene therapy vectors encoding different forms of the PCBP1 (wild-type and mutants) genes in melanoma cells. Lane 1: GFP only; Lane 2: mPCBP1_1+GFP; Lane 3: wPCBP1_0+GFP; Lane 4: PCBP1_2+GFP. Beta-actin is the control.

FIG. 4A shows the comparison of the net cell count difference in Circle Detection Zone (CDZ) as a percentage of migrated cells compared to all cells in the well to demonstrate the migration of transfected melanoma cells. The percentage of migrated cells (per well) in GFP group (11.33) was significantly higher than wPCBP1_0 (wild type PCBP1, also referred to herein as “PCBP1”), mPCBP1_1, and mPCBP1_2. Among these three groups, mPCBP1_2 has lowest percentage of migrated cells in per well (1.82). (*P<0.05).

FIG. 4B shows a cell migration assay. The cell migration assays were read on day 1 and days 5 and were observed under the fluorescent microscope (green filter). The top row shows cell migration of wPCBP1_0 from day 1 and days 5; the middle rows show mPCBP1_2 and mPCBP1_1, and the bottom row shows the GFP group. Column A is cell migration well (white dotted line circle is Circle Detection Zone, or called CDZ) on day 1; column B is cell migration well on day 5 (with CDZ in white dotted line); column C is enlarged CDZ in column B.

FIG. 4C shows the net cell count difference in the Circle Detection Zone (CDZ) Migration Assay as a comparison of the difference among the net cell counts in the CDZ from day 1 to day 5. The melanoma cells were transfected by GFP, wPCBP1_0, mPCBP1_1, or mPCBP1_2.

FIG. 5 shows the fold change of PCBP1 mRNA levels in a breast cancer (BC) Cell line (MCF-7). The graph shows qRT-PCR results of PCBP1 mRNA fold change in PCBP1 (WT/mutants) or GFP-only vector transfected into MCF-7. Compared with the GFP-only group, all PCBP1+GFP groups (WT and mutants) show a significant increase of PCBP1 mRNA expression levels in the breast cancer cell (fold change indicated in figure, *P<0.01). (GFP: Green Fluorescent Protein vector. w PCBP1_0-GFP vector; mPCBP1_1 (one site mutation (c668t)); mPCBP1_2 (two site mutations (a178g & a379g)).

FIG. 6 shows a Western Blot analysis of PRL3 protein levels in transformed breast cancer (MCF-7) cells after treatment with vectors encoding GFP only (lane 1); wPCBP1_0 (lane 2); mPCBP1_1; (lane 3); and mPCBP1_2 (lane 4).

FIG. 7A shows a comparison ratio of the differences for net cells counted in Circle Detection Zone (CDZ) and the fluorescence intensity of the total cells per well.

FIG. 7B shows a breast cancer cell (MCF-7) migration assay. The cell migration assays were read on day 1 and day 5, and were observed under the fluorescent microscope (green filter). (GFP: Green Fluorescent Protein). w PCBP1_0-GFP vector; mPCBP1_1 (one site mutation (c668t))-GFP vector; mPCBP1_2 (two site mutations (a178g & a379g))-GFP vector.

FIG. 7C shows Cell Migration Assay on breast cancer cell (MCF-7) transfected by GFP, wPCBP1_0, mPCBP1_1, or mPCBP1_2.

FIGS. 8A-E show a Breast cancer (MCF-7) dead cell assay after PCBP1 (wild type/mutants) or GFP (control) transfection. Dead cells were measured by removing the supernatant and applying a commercial live/dead cell assay kit. A: breast cancer cell density after GFP transfection and observation by bright field microscopy. B: breast cancer cell density after wild type PCBP1 transfection and observation by bright field microscopy. C: breast cancer cell density after mPCBP1_1 (one site mutation) transfection and observation by bright field microscopy. D: breast cancer cell density after mPCBP1_2 (two site mutations) transfection and observation by bright field microscopy. E: dead cell counts of breast cancer cells by fluorescent intensity measurement using a Microplate reader after transfection by PCBP1 (wild type/mutants) and/or GFP vectors. (GFP: cell transfected by Green Fluorescent Protein. wP: cell transfected by wild type PCBP1. P1: cell transfected by mPCBP1_1 (one site mutation). P2: cell transfected by mPCBP1_2 (two site mutations).

FIGS. 9A-B show PCBP1 expression in pancreatic cancer cells after transfection with one of the compositions of the present disclosure. The graphs show qRT-PCR results of PCBP1 mRNA fold change after a vector containing a wild type PCBP1 (treatment) or GFP (cancer cell) is transfected into pancreatic cancer cell. Compared with GFP group, the PCBP1 group shows significant increase of PCBP1 mRNA level in pancreatic cancer cells (fold change indicated in FIG. 9A). The GFP group also showed a high PRL3 mRNA expression compared to the PCBP1 group (fold change indicated in FIG. 9B).

FIG. 10 shows the fold change of PCBP1 mRNA expression levels in treatment groups with wild-type and mutants of PCBP1 in prostate cancer cell line (DU-145) when compared with GFP group (control). The graph shows the qRT-PCR results demonstrating the PCBP1 mRNA fold expression change in cancer cells (DU-145) transfected with vectors containing either PCBP1 (WT/mutants)+GFP or GFP-only. Compared with the GFP group, all the PCBP1 groups (WT and mutants) demonstrated a significant increase of PCBP1 mRNA levels in the prostate cancer cells (fold change indicated in figure, *P<0.01). (GFP: Green Fluorescent Protein vector. wPCBP1_0-GFP vector; mPCBP1_1 (one site mutation).; mPCBP1_2 (two site mutations).

FIG. 11 shows the measurement of PRL3 mRNA expression levels in glioblastoma cell lines vs. a cortical neuron cell line (HCN-2) by qRT-PCR. Compared with a normal brain cortical cell line, PRL3 mRNA is overexpressed in all six glioblastoma cell lines tested.

FIG. 12 shows a schematic of an AAV vector expressing PCBP1 (wild type or mutant) under the control of a cell-specific promoter. Here, expression of a PCBP1 sequence (wt, mPCBP1_1, or mPCBP1_2) is driven by a cell-specific promoter such as glial fibrillary acidic protein (GFAP) promoter, to treat glioblastoma in the brain. GFP or LacZ are reporter genes used to illustrate co-localization of the therapeutic protein (e.g. PCBP1) and marker protein expression in specific cell types.

FIGS. 13A-B show in vitro detection of transgene expression driven by a specific promoter in glioblastoma (brain) cells. LN229 glioblastoma cells transfected with the phGFAP-LacZ vector were analyzed via immunohistochemistry. Cancer cells (LN299) were observed by bright field microscopy (Panel A). The expressed proteins from the LacZ gene driven by the specific promoter were detected by the anti-β-galactosidase antibody, A11132 (diluted 1:700). (Panel B). Northernnights™ 557-conjugated anti-rabbit antibody (NL004, R&D system) was used as the secondary antibody (dilution 1:200) resulting in a red fluorescent signal. Magnification is 20×.

FIGS. 14A-B shows the in vitro detection of transgene expression driven by a specific promoter in glioblastoma (brain) cells. U87 glioblastoma cells transfected with the phGFAP-LacZ vector were analyzed via immunohistochemistry. Cancer cells (U87) were observed by bright field microscopy (Panel A). The expressed proteins from the LacZ gene driven by the specific promoter were detected by the anti-β-galactosidase antibody, A11132 (diluted 1:700). (Panel B). Northernnights™ 557-conjugated anti-rabbit antibody (NL004, R&D system) was used as the secondary antibody (dilution 1:200) resulting in a red fluorescent signal. Magnification is 20×.

FIGS. 15A-B shows the in vitro detection of transgene expression driven by a specific promoter in kidney cells. 293 FT kidney cells transfected with the phGFAP-LacZ vector were analyzed via immunohistochemistry. 293 FT kidney cells were observed by bright field microscopy (Panel A). The expressed proteins from the LacZ gene driven by the specific promoter were not detected by the anti-β-galactosidase antibody, A11132 (diluted 1:700). (Panel B). Northernnights™ 557-conjugated anti-rabbit antibody (NL004, R&D system) was used as the secondary antibody (dilution 1:200) which would have resulted in a red fluorescent signal if β-galactosidase were expressed in these cells. Magnification is 20×.

FIGS. 16A-B shows the in vitro detection of transgene expression driven by a specific promoter in eye cells. ARPE-19 eye cells transfected with the phGFAP-LacZ vector were analyzed via immunohistochemistry. ARPE-19 eye cells were observed by bright field microscopy (Panel A). The expressed proteins from the LacZ gene driven by the specific promoter were not detected by the anti-β-galactosidase antibody, A11132 (diluted 1:700). (Panel B). Northernnights™ 557-conjugated anti-rabbit antibody (NL004, R&D system) was used as the secondary antibody (dilution 1:200) which would have resulted in a red fluorescent signal if β-galactosidase were expressed in these cells. Magnification is 20×.

FIG. 17 shows the detection of LacZ mRNA expression driven by phGFAP promoter in different cell types by qRT-PCR. LacZ mRNA expression levels in different cells (LN229, U87, ARPE-19, and 293 FT) was monitored after transfection of these cells with a vector containing LacZ under the control of hGFAP. The fold changes were compared with AAV-GFP vectors as control (*p<0.05). These data confirm that the LacZ proteins are expressed in the glioblastoma (brain) cells (e.g. U87 and NL229 cells) under the hGFAP promoter, but not in other cell types (e.g. ARPE-19 and 293 FT cells)

FIGS. 18A-B show the cell viability of glioblastoma cell lines LN229 and U87 cells after transfection with an AAV vector containing either GFP only, wild type PCBP1 (wP), mPCBP1_1 (P1), or mPCBP1_2 (P2). Panel A shows viability of LN229 glioblastoma cells. Panel B shows viability U87 glioblastoma cells. (*p<0.05 vs GFP).

FIGS. 19A-B show the live/dead cell signal in U87 glioblastoma cells after transfection with a control or PCBP-1 containing vector. Cells were transfected with AAV-GFP, AAV-PCBP1 (wP), AAV-PCBP1_1 (P1), or AAV-PCBP1_2 (P2), and cell viability tested after four days. The live cells were stained with Calcein AM (Panel A), and the dead cells were stained with EthD-1 (Panel B). The dead cells were significantly increased after transfection with a PCBP1-containing vector compared with cells transfected with a GFP control vector. *p<0.05

FIGS. 20A-D show the staining of live and dead U87 glioblastoma cells after transfection by AAV-GFP (Panel A), AAV-PCBP1 (Panel B (wP)), AAV-PCBP1_1 (Panel C (Mu1)), or AAV-PCBP1_2 (Panel D (Mu2)).

FIG. 21 shows PRL3 expression levels in U87 glioblastoma cells transfected with either AAV-GFP (lane 1), AAV-PCBP1 (lane 2), AAV-PCBP1_1 (lane 3), or AAV-PCBP1_2 (lane 4). PRL3 protein levels were tested using a Western Blot. Beta actin was used as the internal loading control.

FIGS. 22A-B show the live/dead cell signal in LN229 glioblastoma cells after transfection with a control or PCBP-1 containing vector. Cells were transfected with AAV-GFP, AAV-PCBP1 (wP), AAV-PCBP1_1 (P1), or AAV-PCBP1_2 (P2) and cell viability tested after four days. The live cells were stained with Calcein AM (Panel A), and the dead cells were stained with EthD-1 (Panel B). The dead cells were significantly increased after transfection with a PCBP1-containing vector compared with cells transfected with a GFP control vector. * p<0.05

FIGS. 23A-D show the staining of live and dead LN229 glioblastoma cells after transfection by AAV-GFP (Panel A), AAV-PCBP1 (Panel B (wP)), AAV-PCBP1_1 (Panel C (Mu1)), or AAV-PCBP1_2 (Panel D (Mu2)).

FIG. 24 shows the PRL3 protein levels in LN229 glioblastoma cells transfected with either AAV-GFP (lane 1), AAV-PCBP1 (lane 2), AAV-PCBP1_1 (lane 3), or AAV-PCBP1_2 (lane 4). PRL3 protein levels were tested using a Western Blot. Beta actin was used as the internal loading control.

FIGS. 25A-B show live/dead analysis of the glioblastoma M059J cell line after transfection by AAV-GFP and AAV-PCBP1 and mutants. wP=wild type PCBP1; P1=mPCBP1_1; P2=mPCBP1_2. *p<0.05 compared with GFP.

FIGS. 26A-B show live/dead analysis of the glioblastoma M059K cell line after transfection by AAV-GFP and AAV-PCBP1 and mutants. wP=wild type PCBP1; P1=mPCBP1_1; P2-mPCBP1_2. *p<0.05 compared with GFP.

FIGS. 27A-B show live/dead analysis of the glioblastoma U118MG cell line after transfection by AAV-GFP and AAV-PCBP1 and mutants. wP=wild type PCBP1; P1=mPCBP1_1; P2=mPCBP1_2. *p<0.05 compared with GFP.

FIGS. 28A-B show live/dead analysis of the glioblastoma U138MG cell line after transfection by AAV-GFP and AAV-PCBP1 and mutants. wP=wild type PCBP1; P1=mPCBP1_1; P2=mPCBP1_2. *p<0.05 compared with GFP.

FIG. 29 shows the measurement of pSTAT3 (Y705) protein levels by Western Blot in PCBP 1-transfected U87 glioblastoma cells. Cells were transfected with AAV-GFP (lane 1), AAV-PCBP1 (lane 2), AAV-mPCBP1_1 (lane 3), or AAV-mPCBP1_2 (lane 4). Treatment by all forms of PCBP1 down-regulated STAT3 levels in the glioblastoma cells, with mPCBP1_2 showing the most significant effect. Beta actin served as the internal control.

FIGS. 30A-B show the measurement of pSTAT3 (Y705) protein levels by Western Blot in PCBP1-transfected LN299 glioblastoma cells. Cells were transfected with AAV-GFP (lane 1), AAV-PCBP1 (lane 2), AAV-mPCBP1_1 (lane 3), or AAV-mPCBP1_2 (lane 4). Treatment by all forms of PCBP1 down-regulated STAT3 levels in the glioblastoma cells, with mPCBP1_2 showing the most significant effect. (Panel A). Panel B shows a repeat Western Blot measurement for lanes 1 and 2 in Panel A. Beta actin served as the internal control.

FIGS. 31A-B show the cell proliferation and counts of ovarian SKOV3 cells after transfection with PCBP1 or mutants. GFP=control vector; wild type PCBP=PCBP1; PCBP1 mutation-1=mPCBP1_1; PCBP1-mutation-2=mPCBP1_2. Panel A shows microscopic observation of the cells transfected with either 0.5 μg or 2.0 μg vector per well. Panel B shows the cell numbers as counted by a microplate reader.

FIG. 32 shows the detection of mRNA expression levels of PCBP1 and PRL3 by qRT-PCR in SKOV3 ovarian cells after treatment. The graph shows comparisons of fold changes of mRNA levels (determined by qRT-PCR) among the treatment groups and control. PCBP1 mRNA expression levels show significant increases in all PCBP1 treatment groups, including wild type (11.5 fold increase), mPCBP1_1 (11.3 fold increase), and mPCBP1_2 (11.4 fold increase). In contrast, PRL3 mRNA levels decreased: wild type (1.403 fold decrease), mPCBP1_1 (1.978 fold decrease), and mPCBP1_2 (1.988 fold decrease). GFP=control vector; wild type PCBP=PCBP1; PCBP1 mutation-1=mPCBP1_1; PCBP1-mutation-2=mPCBP1_2.

FIG. 33 shows the effect of wild type and mutant PCBP1 overexpression on the DU145 prostate cancer cell line proliferation. GFP=no PCBP1 treatment group; wP=wild type PCBP1; P1=mPCBP1_1; P2=mPCBP1_2. Overexpression of PCBP1 or the mutant forms significantly inhibited the proliferation of the prostate cancer cells compared to the control.

FIG. 34 shows the measurement of PRL3 protein levels by Western Blot in PCBP1-transfected DU145 prostate cancer cells. Cells were transfected with AAV-GFP (lane 1), AAV-PCBP1 (lane 2), AAV-mPCBP1_1 (lane 3), or AAV-mPCBP1_2 (lane 4). Treatment by all forms of PCBP1 down-regulated PRL3 levels in the prostate cancer cells, with mPCBP1_2 showing the most significant effect. Beta actin served as the internal control.

FIG. 35 shows a cytotoxicity (LDH) assay on a normal brain cell line, HCN-2. The HCN-2 cells were transfected with AAV-GFP, AAV-PCBP1 (wP), AAV-mPCBP1_1 (P1), or AAV-mPCBP1_2 (P2). There was no significant difference among the lactate dehydrogenase (LDH) in the treatment groups compared with the GFP control group (p>0.05), although a downward trend was observed. The cells in the positive control were treated by lysis buffer to elicit a maximum LDH release (left column). The experiments were performed in triplicate.

FIGS. 36A-B show a cytotoxicity (LDH) assay on a normal breast cell line, MCF10A. The MCF10A cells were transfected with AAV-GFP, AAV-PCBP1 (wP), AAV-mPCBP1_1 (P1), or AAV-mPCBP1_2 (P2). There was no significant difference among the lactate dehydrogenase (LDH) in the treatment groups compared with the GFP control group (p>0.05). (Panel A). No significant differences were observed among these four groups as tested by ANOVA (p>0.05). (Panel B). The treatment with wild type PCBP1, mPCBP1_1, or mPCBP1_2 is not cytotoxic to this normal breast cell line.

### DETAILED DESCRIPTION

PRL-3 expression has been found to be elevated in metastatic cancers, including ovarian cancer, prostate, and gastric cancer, and overexpression of PRL-3 correlates with poorer liver cancer prognosis (Bardelli et al. 2003; Polato et al., 2005; Peng et al. 2004). Overexpression of PCBP1 has previously been shown to decrease PRL-3 expression (Wang et al., 2010). The present disclosure provides novel PCBP1 mutations and/or overexpressed PCBP1 which decrease PRL-3 expression to a greater degree than the wild type PCBP1 polypeptide or expression level, and thus decreases cell tumorigenicity and migration.

Thus, the compositions and methods disclosed herein include cells genetically modified (e.g. through transfection or transduction) with a mutant PCBP1 or an overexpressed PCBP1 to reduce or prevent cell migration and/or metastasis. A mutant PCBP1 sequence is incorporated into a vector system for gene therapy to directly prevent, inhibit, decrease, or delay cell migration for the treatment of disease.

### Poly(rC)-Binding Protein 1 (PCBP1)

In some aspects, the present disclosure provides a vector containing the transcription factor Poly(rC)-binding protein 1 (PCBP1, also known as hnRNP-E1 and αCP1) which can inhibit or delay tumorigenesis and/or prevent cell growth or metastasis. In some embodiments, the PCBP1 is a mammalian PCBP1. In some embodiments, the PCBP1 is human PCBP1, mutant, fragment, or variant thereof. In some embodiments, the PCBP1 mutant, fragment, or variant thereof prevent or decrease phosphorylation of the expressed protein. In some embodiments, the PCBP1 is the mutant PCBP1_1 or PCBP1_2 as disclosed herein.

In some embodiments, the PCBP1 decreases cell proliferation or migration. In some embodiments, the PCBP1 decreases cell viability. In some embodiments, the PCBP1 increases cell death. In some embodiments, the PCBP1 affects growth and/or proliferation of immune cells. In some embodiments, the PCBP1 treats inflammation. In some embodiments, the inflammation is associated with cancer.

In some embodiments, the PCBP1 treats cancer. In some embodiments, the PCBP1 treats cancer by changing the expression and/or translation of a cancer biomarker associated with tumorigenesis or metastasis. In some embodiments, the PCBP1 decreases the expression and/or translation of a cancer biomarker associated with tumorigenesis or metastasis. In some embodiments, the cancer biomarker includes, but is not limited to, PRL-3, CD44 variant, E-cadherin, STAT-3, and vimentin. In some embodiments, the cancer biomarker associated with metastasis is PRL-3. In some embodiments the PCBP1 is an overexpressed wild type PCBP1 or a mutated PCBP1, that inhibits mRNA expression of PRL-3. In some embodiments, the PCBP1 is an overexpressed wild type PCBP1 or a mutated PCBP1, that inhibits protein expression of PRL-3.

In some embodiments, the PCBP1 decreases cell migration in genetically modified (e.g. transfected or transduced) cells. In some embodiments, the cell migration is metastasis.

In some embodiments, the mutant PCBP1 has no or decreased phosphorylation. In some embodiments, the mutant PCBP1 has no or decreased PAK phosphorylation. In some embodiments, the mutant PCBP1 inhibits or prevents phosphorylated PCBP1 production.

In some embodiments, the PCBP1 is an overexpressed wild type PCBP1. In some embodiments, the cell is transduced with one or more copy of wild type PCBP1. In some embodiments, the PCBP1 is a mutant. In some embodiments, the mutant PCBP1 is a single mutant. In some embodiments, the mutant PCBP1 is a double mutant. In some embodiments, the mutant PCBP1 is a triple mutant. In some embodiments, the mutant PCBP1 polypeptide comprises a S223L, T60A, and/or T127A mutation or a combination thereof. In some embodiments, the single PCBP1 mutant polypeptide is a S223L mutant (referred to herein as mPCBP1_1). In some embodiments, the double PCBP1 mutant polypeptide is a T60A & T127A mutant (referred to herein as mPCBP1_2).

In some embodiments, the PCBP1 is a nucleic acid. In some embodiments, the nucleic acid is DNA. In some embodiments, the nucleic acid is RNA. In some embodiments, the nucleic acid is mRNA. In some embodiments, the nucleic acid is cDNA. In some embodiments, the PCBP1 contains the nucleic acid sequence of SEQ ID NO: 1 or a mutant thereof. In some embodiments, the PCBP1 is the full-length nucleic acid sequence of SEQ ID NO: 1 or a mutant thereof. In some embodiments, the PCBP1 is a fragment of the nucleic acid sequence of SEQ ID NO: 1 or a mutant thereof. In some embodiments, the PCBP1 is a variant of the nucleic acid sequence of SEQ ID NO: 1 or a mutant thereof. In some embodiments, the mutant PCBP1 nucleic acid comprises a c668t, a178g and/or a379g mutation or a combination thereof. In some embodiments, the single PCBP1 mutant nucleic acid is a c668g mutant (referred to herein as mPCBP1_1). In some embodiments, the double PCBP1 mutant nucleic acid is a a178g & a379g mutant (referred to herein as mPCBP1_2).

Without being bound by theory, each of the KH domains within PCBP1 has been shown to bind to mRNAs of different proteins, and use of one or more KH domain may selectively inhibit the translation of a given protein. In some embodiments, the PCBP1 nucleic acid encodes all three K-homologous (KH) domains. In some embodiments, the PCPB1 nucleic acid encodes two KH domains. In some embodiments, the PCPB1 nucleic acid encodes one KH domain.

Further, mutations in a nuclear localization signal may alter the ability of PCBP1 to translocate into the nucleus, and thus affect later gene expression. In some embodiments, the PCBP1 contains a mutation in one or both nuclear localization signals. In some embodiments, the change in one or both nuclear localization signals inhibits the ability of PCBP1 to translocate into the nucleus.

Phosphorylation also plays a role in the activity of PCBP1 (e.g. nonphosphorylated PCBP1 may lack activity or demonstrate greater activity). In some embodiments, the PCBP1 nucleotide sequence contains a mutation(s) that affects the ability of the PCBP1 polypeptide to be phosphorylated. In some embodiments, the PCBP1 nucleotide sequence mutation(s) prevents PCBP1 polypeptide phosphorylation. In some embodiments, the nonphosphorylated PCBP1 or not fully-phosphorylated PCBP1 polypeptide is not active at wild type levels.

In some embodiments, the PCBP1 polypeptide is expressed from a polycistronic mRNA transcript. In some embodiments, the PCBP1 polypeptide is expressed from a dicistronic mRNA transcript. In some embodiments, the PCBP1 polypeptide is expressed as a fusion protein.

In some embodiments, the PCBP1 nucleotide sequence contains a point mutation(s) relative to wild type PCBP1 (SEQ ID NO: 1). In some embodiments, the PCPB1 nucleotide sequence contains a point mutation(s) that affects phosphorylation. In some embodiments, the PCBP1 point mutation increases phosphorylation. In some embodiments, the PCBP1 point mutation decreases phosphorylation. In some embodiments, the PCBP1 nucleotide sequence contains a mutation(s) that may affect nuclear membrane translocation. In some embodiments, the PCBP1 nucleotide sequence contains a point mutation(s) disclosed in Table 1.

In some embodiments, the point mutation is selected from the group including, but not limited to, G13A, G128C, A178G, T299C, T299A, G326A, A379G, G527A, C652T, C688T, G676A, G700A, G781T, T808G, G814T, A871G, C947T, G1033C, C1034T, G1048C, and/or A127G, or a combination thereof. In some embodiments, the point mutation that increases phosphorylation is selected from the group including, but not limited to, G13A, A178G, T299C, T299A, G326A, A379G, G527A, C652T, G781T, G814T, C947T, G1033C, C1034T, G1048C, and/or G1067T or a combination thereof. In some embodiments, the point mutation that decreases phosphorylation is selected from the group including, but not limited to, C688T, A127G, or G128C, A178G, and/or A379G or a combination thereof. In some embodiments, the point mutation is C668T, A178G, and/or A379G or a combination thereof.

TABLE 1

| Genome_Position | Position_N | Ref_N | Van_N | Position_A | Ref_A | Var_A | Cancer_Type | fraction |
|---|---|---|---|---|---|---|---|---|
| chr2: 70314888- | 13 | G | A | 5 | V | M | DOID: 11054/ | Gain\|Phosphorylation |
| 70314888 | | | | | | | Urinary bladder | |
| | | | | | | | cancer [UBC] | |
| chr2: 70314888- | 13 | G | A | 5 | V | M | DOID: 11054/ | Gain\|Phosphorylation |
| 70314888 | | | | | | | Urinary bladder | |
| | | | | | | | cancer [UBC] | |
| chr2: 70314888- | 13 | G | A | 5 | V | M | DOID: 11054/ | Gain\|Phosphorylation |
| 70314888 | | | | | | | Urinary bladder | |
| | | | | | | | cancer [UBC] | |
| chr2: 70314888- | 13 | G | A | 5 | V | M | DOID: 11054/ | Gain\|Phosphorylation |
| 70314888 | | | | | | | Urinary bladder | |
| | | | | | | | cancer [UBC] | |
| chr2: 70315174- | 299 | T | C | 100 | L | P | DOID: 219/ | Gain\|Phosphorylation |
| 70315174 | | | | | | | Colon cancer | |
| | | | | | | | [Colca] | |
| chr2: 70315174- | 299 | T | A | 100 | L | Q | DOID: 1521/ | Gain\|Phosphorylation |
| 70315174 | | | | | | | Cecum cancer | |
| | | | | | | | [Cecca] | |
| chr2: 70315174- | 299 | T | A | 100 | L | Q | DOID: 219/ | Gain\|Phosphorylation |
| 70315174 | | | | | | | Colon cancer | |
| | | | | | | | [Colca] | |
| chr2: 70315174- | 299 | T | A | 100 | L | Q | DOID: 1993/ | Gain\|Phosphorylation |
| 70315174 | | | | | | | Rectum cancer | |
| | | | | | | | [Recca] | |
| chr2: 70315174- | 299 | T | A | 100 | L | Q | DOID: 1993/ | Gain\|Phosphorylation |
| 70315174 | | | | | | | Rectum cancer | |
| | | | | | | | [Recca] | |
| chr2: 70315174- | 299 | T | A | 100 | L | Q | DOID: 3571/ | Gain\|Phosphorylation |
| 70315174 | | | | | | | Liver cancer | |
| | | | | | | | [Livca] | |
| chr2: 70315174- | 299 | T | A | 100 | L | Q | DOID: 219/ | Gain\|Phosphorylation |
| 70315174 | | | | | | | Colon cancer | |
| | | | | | | | [Colca] | |
| chr2: 70315174- | 299 | T | A | 100 | L | Q | DOID: 219/ | Gain\|Phosphorylation |
| 70315174 | | | | | | | Colon cancer | |
| | | | | | | | [Colca] | |
| chr2: 70315174- | 299 | T | C | 100 | L | P | DOID: 219/ | Gain\|Phosphorylation |
| 70315174 | | | | | | | Colon cancer | |
| | | | | | | | [Colca] | |
| chr2: 70315174- | 299 | T | A | 100 | L | Q | DOID: 1993/ | Gain\|Phosphorylation |
| 70315174 | | | | | | | Rectum cancer | |
| | | | | | | | [Recca] | |
| chr2: 70315174- | 299 | T | A | 100 | L | Q | DOID: 219/ | Gain\|Phosphorylation |
| 70315174 | | | | | | | Colon cancer | |
| | | | | | | | [Colca] | |
| chr2: 70315201- | 326 | G | A | 109 | C | Y | DOID: 5041/ | Gain\|Phosphorylation |
| 70315201 | | | | | | | Esophageal | |
| | | | | | | | cancer [EC] | |
| chr2: 70315402- | 527 | G | A | 176 | G | E | DOID: 4159/ | Gain\|Phosphorylation |
| 70315402 | | | | | | | Skin cancer | |
| | | | | | | | [Skica] | |
| chr2: 70315527- | 652 | C | T | 218 | P | S | DOID: 11054/ | Gain\|Phosphorylation |
| 70315527 | | | | | | | Urinary bladder | |
| | | | | | | | cancer [UBC] | |
| chr2: 70315527- | 652 | C | T | 218 | P | S | DOID: 11054/ | Gain\|Phosphorylation |
| 70315527 | | | | | | | Urinary bladder | |
| | | | | | | | cancer [UBC] | |
| chr2: 70315543- | 668 | C | T | 223 | S | L | DOID: 11054/ | Loss\|Phosphorylation |
| 70315543 | | | | | | | Urinary bladder | |
| | | | | | | | cancer [UBC] | |
| chr2: 70315543- | 668 | C | T | 223 | S | L | DOID: 11054/ | Loss\|Phosphorylation |
| 70315543 | | | | | | | Urinary bladder | |
| | | | | | | | cancer [UBC] | |
| chr2: 70315543- | 668 | C | T | 223 | S | L | DOID: 11054/ | Loss\|Phosphorylation |
| 70315543 | | | | | | | Urinary bladder | |
| | | | | | | | cancer [UBC] | |
| chr2: 70315656- | 781 | G | T | 261 | D | Y | DOID: 1793/ | Gain\|Phosphorylation |
| 70315656 | | | | | | | Pancreatic | |
| | | | | | | | cancer [PACA] | |
| chr2: 70315689- | 814 | G | T | 272 | A | S | DOID: 1993/ | Gain\|Phosphorylation |
| 70315689 | | | | | | | Rectum cancer | |
| | | | | | | | [Recca] | |
| chr2: 70315689- | 814 | G | T | 272 | A | S | DOID: 1993/ | Gain\|Phosphorylation |
| 70315689 | | | | | | | Rectum cancer | |
| | | | | | | | [Recca] | |
| chr2: 70315689- | 814 | G | T | 272 | A | S | DOID: 1993/ | Gain\|Phosphorylation |
| 70315689 | | | | | | | Rectum cancer | |
| | | | | | | | [Recca] | |
| chr2: 70315822- | 947 | C | T | 316 | A | V | DOID: 10534/ | Gain\|Phosphorylation |
| 70315822 | | | | | | | Stomach cancer | |
| | | | | | | | [Stoca] | |
| chr2: 70315822- | 947 | C | T | 316 | A | V | DOID: 10534/ | Gain\|Phosphorylation |
| 70315822 | | | | | | | Stomach cancer | |
| | | | | | | | [Stoca] | |
| chr2: 70315908- | 1033 | G | C | 345 | A | P | DOID: 1319/ | Gain\|Phosphorylation |
| 70315908 | | | | | | | Brain cancer | |
| | | | | | | | [Braca] | |
| chr2: 70315908- | 1033 | G | C | 345 | A | P | DOID: 1319/ | Gain\|Phosphorylation |
| 70315908 | | | | | | | Brain cancer | |
| | | | | | | | [Braca] | |
| chr2: 70315909- | 1034 | C | T | 345 | A | V | DOID: 363/ | Gain\|Phosphorylation |
| 70315909 | | | | | | | Uterine cancer | |
| | | | | | | | [Uteca] | |
| chr2: 70315909- | 1034 | C | T | 345 | A | V | DOID: 363/ | Gain\|Phosphorylation |
| 70315909 | | | | | | | Uterine cancer | |
| | | | | | | | [Uteca] | |
| chr2: 70315909- | 1034 | C | T | 345 | A | V | DOID: 363/ | Gain\|Phosphorylation |
| 70315909 | | | | | | | Uterine cancer | |
| | | | | | | | [Uteca] | |
| chr2: 70315923- | 1048 | G | C | 350 | E | Q | DOID: 1324/ | Gain\|Phosphorylation |
| 70315923 | | | | | | | Lung cancer | |
| | | | | | | | [Lunca] | |
| chr2: 70315909 | 127/128 | A/G | G/C | 43 | S | A | Breast cancer^{a} | Loss\|Phosphorylation |
| chr2: 70315909 | 178 | A | G | 60 | T | A | Breast cancer^{b} | Loss\|Phosphorylation |
| chr2: 70315909 | 379 | A | G | 127 | T | A | Breast cancer^{b} | Loss\|Phosphorylation |

^{a}Brown, A (2016);
^{b}Qingchang Meng, et al.

In some embodiments, the disclosure provides mimetics, analogs, derivatives, variants, or mutants of PCBP1 (SEQ ID NO: 1). In some embodiments, the mimetic, analog, derivative, variant, or mutant contains one or more nucleic acid substitutions compared to the nucleic acid sequence of the native PCBP1. In some embodiments, one to 20 nucleic acids are substituted. In some embodiments, the mimetic, analog, derivative, variant, or mutant contains about 1, about 2, about 3, about 4, about 5, about 6, about 7, about 8, about 9, or about 10 nucleic acid substitutions compared to the nucleic acid sequence of the native PCBP1 (e.g. SEQ ID NO: 1). In some embodiments, the mimetic, analog, derivative, variant, or mutant contains one or more nucleic acid deletions compared to the nucleic acid sequence of the native PCBP1 (e.g. SEQ ID NO: 1). In some embodiments, the mimetic, analog, derivative, variant, or mutant comprises SEQ ID NO: 3 (mPCBP1_1 (c668t)). In some embodiments, the mimetic, analog, derivative, variant, or mutant comprises SEQ ID NO: 5 (mPCBP1_2 (a178g & a379g).

In some embodiments, one to 20 nucleic acid residues are deleted compared to the nucleic acid sequence of the native PCBP1 (e.g. SEQ ID NO: 1). In some embodiments, the mimetic, analog, derivative, variant, or mutant has about 1, about 2, about 3, about 4, about 5, about 6, about 7, about 8, about 9, or about 10 nucleic acid residue deletions compared to the nucleic acid sequence of the native PCBP1 (e.g. SEQ ID NO: 1). In some embodiments, one to ten nucleic acid residues are deleted at either terminus compared to the nucleic acid sequence of the native PCBP1(e.g. SEQ ID NO: 1). In some embodiments, one to ten nucleic acid residues are deleted from both termini compared to the nucleic acid sequence of the native PCBP1. In some embodiments, the nucleic acid sequence of the mimetic, analog, derivative, variant, or mutant is at least about 70% identical to the nucleic acid sequence of the native PCBP1. In some embodiments, the nucleic acid sequence of the mimetic, analog, derivative, variant, or mutant is about 70%, about 80%, about 85%, about 90%, about 95%, about 96%, about 97%, about 98%, or about 99% identical to the nucleic acid sequence of the native PCBP1 (e.g. SEQ ID NO: 1). Percentage identity can be calculated using the alignment program EMBOSS Needle.

In some embodiments, the PCBP1 is a polypeptide. In some embodiments, the PCBP1 contains the amino acid sequence of SEQ ID NO: 2. In some embodiments, the PCBP1 is the full-length amino acid sequence of SEQ ID NO: 2. In some embodiments, the PCBP1 is a fragment of the amino acid sequence of SEQ ID NO: 2. In some embodiments, the PCB1 is a variant of the amino acid sequence of SEQ ID NO: 2. In some embodiments, the PCBP1 variant includes the amino acid sequence of SEQ ID NO: 4 (mPCBP1_1 (S223L)). In some embodiments, the PCBP1 variant includes the amino acid sequence of SEQ ID NO: 6 (mPCBP1_2T60A & T127A). In some embodiments, the PCBP1 is a functional fragment or variant of the amino acid sequence of SEQ ID NO: 2. In some embodiments, the PCBP1 polypeptide contains all three K-homologous (KH) domains. In some embodiments, the PCPB1 polypeptide contains two KH domains. In some embodiments, the PCPB1 polypeptide contains one KH domain.

In some embodiments, the PCBP1 polypeptide contains one mutated K-homologous (KH) domain. In some embodiments, the PCBP1 polypeptide contains two mutated K-homologous (KH) domains. In some embodiments, the PCBP1 polypeptide contains three mutated K-homologous (KH) domains. In some embodiments, the mutation in the one or more KH domains effects RNA binding.

In some embodiments, the PCBP1 polypeptide sequence contains an amino acid mutation relative to wild type PCBP1 (SEQ ID NO: 2). In some embodiments, the PCPB1 polypeptide sequence contains an amino acid mutation that affects phosphorylation. In some embodiments, the amino acid mutation is selected from the group including, but not limited to, V5M, S43A, T60A, L100P, L100Q, C109Y, A127G, G128C, T237A, G176E, P218S, G226R, D234N, D261Y, Y270D, A272S, 1291V, A316V, A345P, A345V, E350Q, S356I, or a combination thereof. In some embodiments, the mutation that increases phosphorylation is selected from the group including, but not limited to, V5M, L100P, L100Q, C109Y, G176E, D261Y, A272S, A316V, A345P, A345V, and/or E350Q or a combination thereof. In some embodiments, the mutation that decreases phosphorylation is selected from the group including, but not limited to, S43A, T60A, T127A, and/or S223L, or a combination thereof. In some embodiments, the amino acid mutation is T60A, S223L, S43A, and/or T127A.

In some embodiments, the disclosure provides mimetics, analogs, derivatives, variants, or mutants of PCBP1 (SEQ ID NO:2). In some embodiments, the mimetic, analog, derivative, variant, or mutant contains one or more amino acid substitutions compared to the amino acid sequence of the native PCBP1. In some embodiments, more than 20 amino acids are substituted. In some embodiments, one to 20 amino acids are substituted. In some embodiments, the mimetic, analog, derivative, variant, or mutant contains about 1, about 2, about 3, about 4, about 5, about 6, about 7, about 8, about 9, or about 10 amino acid substitutions or more compared to the amino acid sequence of the native PCBP1 (SEQ ID NO:2). In some embodiments, the mimetic, analog, derivative, variant, or mutant comprises SEQ ID NO: 4. In some embodiments, the mimetic, analog, derivative, variant, or mutant comprises SEQ ID NO: 6.

In some embodiments, the mimetic, analog, derivative, variant, or mutant contains one or more amino acid deletions compared to the amino acid sequence of the native therapeutic peptide agent. In some embodiments, more than 20 amino acids are deleted. In some embodiments, one to 20 amino acids are deleted compared to the amino acid sequence of the native protein agent. In some embodiments, the mimetic, analog, derivative, variant, or mutant has about 1, about 2, about 3, about 4, about 5, about 6, about 7, about 8, about 9, or about 10 amino acid deletions or more compared to the amino acid sequence of the native PCBP1 (SEQ ID NO:2). In some embodiments, one to ten amino acids are deleted at either terminus compared to the amino acid sequence of the native PCBP1 (SEQ ID NO:2). In some embodiments, one to ten amino acids are deleted from both termini compared to the amino acid sequence of the native PCBP1 (SEQ ID NO:2). In some embodiments, the amino acid sequence of the mimetic, analog, derivative, variant, or mutant is at least about 70% identical to the amino acid sequence of the native PCBP1 (SEQ ID NO:2). In some embodiments, the amino acid sequence of the mimetic, analog, derivative, variant, or mutant is about 70%, about 80%, about 85%, about 90%, about 95%, about 96%, about 97%, about 98%, or about 99% identical to the amino acid sequence of the native PCBP1 (SEQ ID NO:2). In some embodiments, the amino acid sequence of the mimetic, analog, derivative, variant, or mutant is about 70%, about 80%, about 85%, about 90%, about 95%, about 96%, about 97%, about 98%, or about 99% identical to the amino acid sequence of the native PCBP1 (SEQ ID NO:2) and retains all or most of the biological activity of the native PCBP1. In some embodiments, the amino acid sequence of the mimetic, analog, derivative, variant, or mutant is about 70%, about 80%, about 85%, about 90%, about 95%, about 96%, about 97%, about 98%, or about 99% identical to the amino acid sequence of the native PCBP1 (SEQ ID NO:2) and has reduced or altered activity compared with the native PCBP1.

In some embodiments, the amino acid sequence of the mimetic, analog, derivative, variant, or mutant is about 70%, about 80%, about 85%, about 90%, about 95%, about 96%, about 97%, about 98%, or about 99% identical to the amino acid sequence of one or more domains of the native PCBP1 (SEQ ID NO:2). In some embodiments, the amino acid sequence of the mimetic, analog, derivative, variant, or mutant is about 70%, about 80%, about 85%, about 90%, about 95%, about 96%, about 97%, about 98%, or about 99% identical to the amino acid sequence of one or more domains of the native PCBP1 (SEQ ID NO:2) and retains all or most of the biological activity of the native PCBP1. In some embodiments, the amino acid sequence of the mimetic, analog, derivative, variant, or mutant is about 70%, about 80%, about 85%, about 90%, about 95%, about 96%, about 97%, about 98%, or about 99% identical to the amino acid sequence of one or more domains of the native PCBP1 (SEQ ID NO:2) and has reduced or altered activity compared with the native PCBP1. Percentage identity can be calculated using the alignment program EMBOSS Needle. The following default parameters may be used for Pairwise alignment: Protein Weight Matrix=BLOSUM62; Gap Open=10; Gap Extension=0.1.

### Vectors and Plasmids

Efficient delivery of the therapeutic gene to the target tissue or cell is the most significant hurdle for successful gene therapy. Since naked DNA is rapidly cleared or degraded in vivo by phagocytic immune cells or extracellular nucleases, a means of protecting the transgene may be desired. Furthermore, a vehicle for effecting tissue or cell entry is also required, due to the poor efficiency of spontaneous DNA uptake. Thus, DNA is normally combined with a gene delivery vehicle of some type, commonly known as a vector, to protect and mediate effective tissue or cell entry of the gene of interest.

Gene delivery systems can be grouped into non-biological (e.g. chemical and physical approaches of introducing plasmid DNA to mammalian cells) or biological (e.g. viruses and bacteria). Non-viral gene delivery systems normally involve the transfer genes carried on plasmid DNA. Plasmids employed do not generally replicate in mammalian cells.

Most commonly, recombinant viruses or naked DNA or DNA complexes are used. For example, viruses can be modified in the laboratory to provide vectors that carry corrected, therapeutic DNA into cells, where it can be integrated into the genome to alter abnormal gene expression and correct genetic disease. Alternatively, the vector may remain extrachromosomal and be expressed transiently.

In some aspects, the present disclosure provides methods of gene therapy using viral vectors. Viruses that may be used in gene therapy include, but are not limited to, lentiviruses, retroviruses, adenoviruses, adeno-associated viruses, replication-competent vectors, vaccinia virus, and the herpes simplex virus. Viral vectors that may be used in gene therapy include, but are not limited to, lentiviral, retrovirual, adenoviruses, adeno-associated viruses vectors (AAV), replication-competent vectors, vaccinia virus vectors, and the herpes simplex virus vectors.

In some aspects, the present disclosure provides methods of gene therapy using non-viral vectors. In some aspects, the present disclosure provides methods of gene therapy using bacterial vectors. In some embodiments, the gene therapy method involves injection of naked nucleic acids (e.g. DNA or RNA). This may be performed using any appropriate means known in the art. In some aspects, the present disclosure provides methods of gene therapy using bacterial delivery systems. In some embodiments, the bacterial cells are live, attenuated, or killed. In some embodiments, the bacterial delivery system exploits a cell's ability to adhere to a mammalian cell or a secretion system to deliver the nucleic acid and/or proteins to the target mammalian cell. In some embodiments, the bacterial delivery system is selected from, but not limited to, Salmonella typhi, Bifidobacterium spp., Salmonella choleraesuis, Vibrio cholera, Listeria monocytogenes, Escherichia coli, Streptococcus pyogenes, and Serratia marcescens.

In some aspects, the present disclosure provides methods of gene therapy using non-viral and non-bacterial vectors. In some embodiments, the non-viral and non-bacterial vector is a eukaryotic vector. In some embodiments, the eukaryotic vector includes a transposon system, a CRISPR system, a Zinc-finger nuclease system, or a TALEN (transcription-activator-like effector nuclease). In some embodiments, the transposon system is selected from, but not limited to, the Sleeping Beauty or piggyBac or transposon system. In some embodiments, other methods for nucleic acid delivery may be used such as arginine-rich peptides or sonoporation.

In some aspects, the present disclosure provides methods of gene therapy using nanoparticles. In some embodiments, the nanoparticle is a lipid-based nanoparticle. In some embodiments, the lipid-based nanoparticle is a solid lipid-nanoparticle (SLN). In some embodiments, the lipid-based nanoparticle is a non-structured lipid carrier (NLC). In some embodiments, the nanoparticle is a polymer-based nanoparticle. In some embodiments, the polymer-based nanoparticle is a nanosphere or a nanocapsule.

Plasmid DNA-based vectors are commonly used in gene therapy and can accommodate large segments of DNA and allows the manipulation of a variety of regulatory elements that impact gene transfer and expression. At its most basic, an expression plasmid contains an expression cassette and backbone. The expression cassette is a transcriptional unit containing the gene or genes of interest and any regulatory sequences required for expression in the target cells. The backbone may contain a selectable marker (e.g. an antibiotic resistance gene or an auxotrophic selection gene) and an origin of replication required for the production of the plasmid in bacteria.

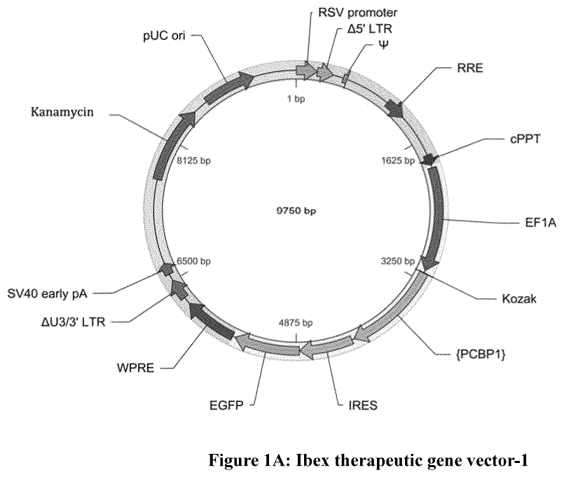

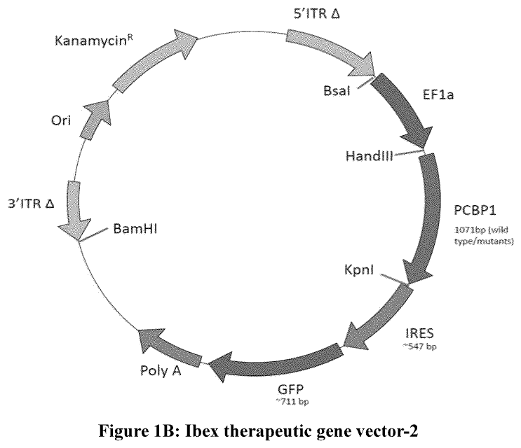

Any appropriate plasmid may be used in the methods disclosed herein. In some embodiments, the plasmid is a lentiviral vector. In some embodiments, the plasmid is an adeno-associated virus (AAV) plasmid. In some embodiments, representative plasmids are disclosed in FIGS. 1A-B. Any appropriate promoter may be used to drive expression of the genes in the expression cassette.

In some embodiments, the plasmid contains a retroviral promoter. In some embodiments, the plasmid contains a viral promoter. In some embodiments, the plasmid contains a mammalian promoter. In some embodiments, the mammalian promoter is a human promoter. In some embodiments, the promoter is tissue or cell specific. In some embodiments, the tissue specific promoter is selected from human transducing alpha-subunit promoter, GANT2, VMD2, human IRBP, K14, IRS2, and glial fibrillary acidic protein (GFAP) promoters. The promoter selected may be used as a chimeric promoter (e.g. with the interphotoreceptor retinoid binding protein (IRBP) promoter). In some embodiments, the tissue-or cell-specific promoter is specific for pancreatic cells, pancreatic beta cells, skin cells, keratinocytes, photoreceptor cells, epithelial cells, endothelial cells, and/or cancer cells (e.g. breast cancer cells, prostate cancer cells, leukemia cells, lymphoma cells, neural cancer cells, glioblastoma, etc.). In some embodiments, the promoter is specific for retinal or eye tissue or cells. In some embodiments, the promoter is specific for hematopoietic cells. In some embodiments, the promoter is specific for liver tissue or cells. In some embodiments, the promoter is specific for lung tissue or cells. In some embodiments, the promoter is specific for muscle tissue or cells. In some embodiments, the promoter is specific for HIV-infected cells.

In some embodiments, the promoter is an inducible promoter. In some embodiments, the promoter is induced by any appropriate composition or stimulus, including, but not limited to, doxycycline, tetracycline, IPTG, ecdysone, or rapamycin. In some embodiments, the inducible promoter is selected from any known to those skilled in the art, including but not limited to, a TRE3G, tetracycline, Lac, ecdysone, and rapamycin promoter. In some embodiments, the promoter is constitutive. In some embodiments, the promoter is a synthetic promoter or contains enhancer elements. In some embodiments, the promoter is a hybrid promoter. In some embodiments, the hybrid promoter contains regulatory regions of a gene.

In some embodiments, the promoter is selected from the group including, but not limited to, Ef1a, CAG, sv40, CMV, RSV, Oct4, Rex1, Nanog, GANT2, VMD2, hIRBP, TET promoter, CAAT Box, GC Box, GT-1 motif, I-box, AT-rich sequence, RBCS1, TRE3G, GAL1, Lap267, Rapamycin, CD11a, CD11b, CD18, Beta-globin promoter/LCR, Immunoglobulin promoter, PEPCK promoter, Albumin promoter, hAAT, SPC, SP-A, MCK, VLC1, HIV-LTR, Tat/Rev-responsive elements, Tat-inducible element, and FMR1.

In some embodiments, the plasmid contains all of the genes to be introduced via gene therapy. In some embodiments, the plasmid contains a PCBP1. In some embodiments, the PCBP1 is a mutant PCBP1. In some embodiments, the mimetic, analog, derivative, variant, or mutant comprises SEQ ID NO: 3. In some embodiments, the mimetic, analog, derivative, variant, or mutant comprises SEQ ID NO: 5. In some embodiments, the PCBP1 mimetic, analog, derivative, variant, or mutant encodes a polypeptide comprising the amino acid sequence of SEQ ID NO: 4or SEQ ID NO: 6.

In some embodiments, introduction of the vectors of the disclosure increase gene copy number in the cell. In some embodiments, introduction of the vectors of the disclosure increase gene expression in the cell. In some embodiments, introduction of the vectors of the disclosure increase polypeptide expression in the cell. In some embodiments, the gene is PCBP1 or a mutant thereof.

### Diseases and Disorders

The compositions and methods disclosed herein may be employed on any appropriate cell or tissue type. In some embodiments, the methods disclosed herein are performed in vitro. In some embodiments, the methods disclosed herein are performed ex vivo. In some embodiments, the methods disclosed herein are performed on isolated cells. In some embodiments, the methods disclosed herein are performed on cell culture. In some embodiments, the isolated cells or cell culture cells are taken from the subject and then implanted after transfection or transduction. In some embodiments, the cells are cancer cells.

In some embodiments, the compositions and methods disclosed herein are employed in vivo. In some embodiments, the methods disclosed herein are employed in situ. In some embodiments, the methods disclosed herein are used to decrease cell migration in any appropriate part of the body, including, but not limited to, the eye, retina, heart, blood, white blood cell, red blood cell, platelet, vitreous humor, sclera, retina, iris, cornea, skeletal muscle, cardiac muscle, smooth muscle, cartilage, tendon, bone, epidermis, organ, liver, heart, kidney, lung, stomach, gastrointestinal tract, colon, bladder, ovary, testes, pancreas, bone marrow, brain, neuron, and/or gland.

The compositions and methods disclosed herein may be used to treat, prevent, ameliorate, or delay any appropriate disease. For example, diseases that may be treated, prevented, ameliorated, or delayed are characterized by cell migration.

In some embodiments, the compositions and methods disclosed herein may be used to treat cancer or uncontrolled cell growth. In some embodiments, the compositions and methods disclosed herein are used to prevent, inhibit, ameliorate, or decrease metastasis, or uncontrolled cell migration. In some embodiments, the cancer is a solid cancer. In some embodiments, the cancer is a non-solid cancer. In some embodiments, the disclosure relates to cancers including, but not limited to, acute lymphoblastic leukemia (ALL), acute myeloid leukemia (AML), adrenocortical carcinoma, AIDS-related cancers, anal cancer, appendix cancer, astrocytoma (e.g. childhood cerebellar or cerebral), basal-cell carcinoma, bile duct cancer, bladder cancer, bone tumor (e.g. osteosarcoma, malignant fibrous histiocytoma), brainstem glioma, brain cancer, brain tumors (e.g. glioblastoma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumors, central nervous system lymphomas, cerebellar astrocytoma, cervical cancer, chronic lymphocytic leukemia (CLL), chronic myelogenous leukemia (CML), chronic myeloproliferative disorders, colon cancer, cutaneous t-cell lymphoma, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer, gallbladder cancer, gastric (stomach) cancer, gastrointestinal stromal tumor (GIST), germ cell tumor (e.g. extracranial, extragonadal, ovarian), gestational trophoblastic tumor, gliomas (e.g. brain stem, cerebral astrocytoma, visual pathway and hypothalamic), gastric carcinoid, head and neck cancer, heart cancer, hepatocellular (liver) cancer, hypopharyngeal cancer, hypothalamic and visual pathway glioma, intraocular melanoma, islet cell carcinoma (endocrine pancreas), kidney cancer (renal cell cancer), laryngeal cancer, leukemias (e.g. acute lymphocytic leukemia, acute myelogenous leukemia, chronic lymphocytic leukemia, chronic myeloid leukemia, hairy cell), lip and oral cavity cancer, liposarcoma, liver cancer, lung cancer (e.g. non-small cell, small cell), lymphoma (e.g. AIDS-related, Burkitt, cutaneous T-cell Hodgkin, non-Hodgkin, primary central nervous system), medulloblastoma, melanoma, Merkel cell carcinoma, mesothelioma, metastatic squamous neck cancer, mouth cancer, multiple endocrine neoplasia syndrome, multiple myeloma, mycosis fungoides, myelodysplastic syndromes, myelodysplastic/myeloproliferative diseases, myelogenous leukemia, myeloid leukemia, myeloid leukemia, myeloproliferative disorders, chronic, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma, ovarian cancer, pancreatic cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, retinoblastoma, rhabdomyosarcoma, salivary gland cancer, sarcoma (e.g. Ewing family, Kaposi, soft tissue, uterine), Sézary syndrome, skin cancer (e.g. nonmelanoma, melanoma, Merkel cell), small cell lung cancer, small intestine cancer, soft tissue sarcoma, squamous cell carcinoma, squamous neck cancer, stomach cancer, supratentorial primitive neuroectodermal tumor, t-cell lymphoma, testicular cancer, throat cancer, thymoma and thymic carcinoma, thyroid cancer, trophoblastic tumors, ureter and renal pelvis cancers, urethral cancer, uterine cancer, uterine sarcoma, vaginal cancer, visual pathway and hypothalamic glioma, vulvar cancer, Waldenström macroglobulinemia, and Wilms tumor. In some embodiments, the cells transfected or transduced with the compositions of the disclosure are obtained from one or more of these types of cancer.

### Administration

The compositions of the present disclosure may be administered via any appropriate means. In some embodiments, the nucleic acid route of administration is transdermal, injection, intramuscular, subcutaneous, oral, nasal, intra-vaginal, rectal, transmucosal, enteric, parenteral, topical, epidural, intracerebral, Intracerebroventricular, intra-arterial, antra-articular, intradermal, intra-lesion, intraocular, intraosseous, intraperitoneal, intrathecal, intrauterine, intravenous, intra-vesical infusion, or intravitreal.

In some embodiments, the compositions or cells transfected or transduced with the compositions using the methods disclosed herein are administered to a patient. In some embodiments, the compositions or cells transfected or transduced with the compositions created using the methods disclosed herein are administered to a patient to treat, prevent, ameliorate, or delay the onset of a disease or disorder. In some embodiments, administration of the compositions disclosed herein prevent, decrease, ameliorate, or delay cell migration in the subject. In some embodiments, administration of the compositions disclosed herein prevent, decrease, ameliorate, or delay cell migration in the subject and treat, prevent, ameliorate, or delay the onset of a disease or disorder. In some embodiments, the disease or disorder is a cancer or metastatic cancer. In some embodiments, administration of the compositions disclosed herein decrease tumor mass/burden in a subject.

In some embodiments, the vectors, nucleic acids, or cells disclosed herein are administered once to a patient. In some embodiments, the vectors, nucleic acids, or cells disclosed herein are administered about 2 times, about 3 time, about 4 times, about 5 times, about 6 times, about 7 times, about 8 times, about 9 times, about 10 times, about 20 times, about 40 times, or more to a patient. Vectors, nucleic acids, or cells disclosed herein are administered until disease or disorder symptoms improve.

In some embodiments, administration of the vectors or nucleic acids disclosed herein prevent, ameliorate, decrease, or delay cell migration or proliferation in a treated patient compared to an untreated patient or the same patient before treatment. In some embodiments, cell migration or proliferation is prevented, ameliorated, decreased, or delayed in the treated patient between day 1 and year 10. In some embodiments, administration of the plasmids or vectors disclosed herein prevent, ameliorate, decrease, or delay cell migration or proliferation at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with cell migration or proliferation in an untreated patient or the same patient before treatment. In some embodiments, administration of the nucleic acids or vectors disclosed herein prevents, ameliorates, decreases, or delays cell migration or proliferation for about 1 day, about 1 week, about 1 month, about 2 months, about 3 months, about 4 months, about 5 months, about 6 months, about 1 year, about 2 years, about 5 years, or about 10 years, or more compared with cell migration or proliferation in an untreated patient or the same patient before treatment.

In some embodiments, cell migration or proliferation is decreased by about 1%, about 5%, about 10%, about 20%, about 30%, about 40%, about 50%, about 60%, about 70%, about 80%, about 90%, or about 100% compared with controls or patients or in vitro cells treated with other cell migration or proliferation inhibition methods. In some embodiments, administration of the nucleic acids, vectors, or cells disclosed herein reduces cell migration or proliferation by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients or in vitro cells treated with other cell migration or proliferation prevention/inhibition methods. In some embodiments, administration of the nucleic acids, vectors, or cells disclosed herein reduces cell migration or proliferation by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with controls or patients or in vitro cells treated with other cell migration or proliferation prevention/inhibition methods.

In some embodiments, administration of the vectors or nucleic acids disclosed herein reduce tumor mass or burden in a treated patient compared to an untreated patient or the same patient before treatment. In some embodiments, tumor mass/burden is decreased in the treated patient between day 1 and year 10. In some embodiments, administration of the plasmids or vectors disclosed herein reduces tumor mass or burden at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with tumor mass or burden in an untreated patient or the same patient before treatment. In some embodiments, administration of the nucleic acids or vectors disclosed herein reduces tumor mass/burden for about 1 day, about 1 week, about 1 month, about 2 months, about 3 months, about 4 months, about 5 months, about 6 months, about 1 year, about 2 years, about 5 years, or about 10 years, or more compared with tumor mass or burden in an untreated patient or the same patient before treatment.

In some embodiments, tumor mass or burden is decreased by about 1%, about 5%, about 10%, about 20%, about 30%, about 40%, about 50%, about 60%, about 70%, about 80%, about 90%, or about 100% compared with controls or patients or in vitro cells treated with other tumor mass or burden reduction methods. In some embodiments, administration of the nucleic acids, vectors, or cells disclosed herein reduces tumor mass or burden by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients or in vitro cells treated with other tumor mass or burden reduction methods. In some embodiments, administration of the nucleic acids, vectors, or cells disclosed herein reduces tumor mass or burden by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with controls or patients or in vitro cells treated with other tumor mass or burden reduction methods.

In some embodiments, the vectors, nucleic acids, or cells disclosed herein reduce cancer biomarker expression in treated cells in vitro. In some embodiments, administration of the vectors, nucleic acids, or cells disclosed herein reduces cancer biomarker expression in a treated patient. In some embodiments, administration of the vectors, nucleic acids, or cells disclosed herein reduce cancer biomarker expression in a treated patient or in vitro cells between day 1 and year 10. In some embodiments, administration of the nucleic acids, vectors, or cells disclosed herein reduces cancer biomarker expression at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients or in vitro cells treated with other cancer biomarker expression inhibition methods. In some embodiments, administration of the plasmids, vectors, or cells disclosed herein reduces cancer biomarker expression for about 1 day, 2 days, 3 days, 4 days, 5 days, 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with controls or patients or in vitro cells treated with other cancer biomarker expression inhibition methods.

In some embodiments, cancer biomarker expression is decreased by about 1%, about 5%, about 10%, about 20%, about 30%, about 40%, about 50%, about 60%, about 70%, about 80%, about 90%, or about 100% compared with controls or patients or in vitro cells treated with other cancer biomarker expression inhibition methods. In some embodiments, administration of the nucleic acids, vectors, or cells disclosed herein reduces cancer biomarker expression by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients or in vitro cells treated with other cancer biomarker expression inhibition methods. In some embodiments, administration of the nucleic acids, vectors, or cells disclosed herein reduces cancer biomarker expression by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with controls or patients or in vitro cells treated with other cancer biomarker expression inhibition methods.

In some embodiments, administration of the vectors, nucleic acids, or cells disclosed herein reduces metastasis in an in vitro or ex vivo organ or tissue. In some embodiments, administration of the vectors, nucleic acids, or cells disclosed herein reduces metastasis in a tissue, organ, or a treated patient. In some embodiments, administration of the vectors, nucleic acids, or cells disclosed herein reduces metastasis in a treated patient or in vitro or ex vivo organ or tissue between day 1 and year 10. In some embodiments, administration of the nucleic acids, vectors, or cells disclosed herein reduces metastasis at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients, or in vitro or ex vivo organ or tissue treated with other metastasis inhibition methods. In some embodiments, administration of the nucleic acids, vectors, or cells disclosed herein reduces metastasis or tumorigenesis for about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with controls or patients, or in vitro or ex vivo organ or tissue treated with other metastasis inhibition methods.

In some embodiments, metastasis is decreased by about 1%, about 5%, about 10%, about 20%, about 30%, about 40%, about 50%, about 60%, about 70%, about 80%, about 90%, or about 100% compared with controls or patients treated with other metastasis inhibition methods. In some embodiments, administration of the nucleic acids, vectors, or cells disclosed herein reduces metastasis by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients or in vitro or ex vivo organ or tissue treated with other metastasis inhibition methods. In some embodiments, administration of the nucleic acids, vectors, or cells disclosed herein reduces metastasis by about 1%, about 5%, about 10%, about 20%, about 30%, about 40%, about 50%, about 60%, about 70%, about 80%, about 90%, or about 100% for about 1 day, about 2 days, about 3 days, about 4 days, about 5 days, about 6 days, about day 1, about day 2, about day 3, about day 4, about day 5, about day 6, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared controls or patients or in vitro or ex vivo organ or tissue treated with other metastasis inhibition methods.

In some embodiments, administration of the vectors, nucleic acids, or cells disclosed herein increases cell death in a tissue, organ, or a treated patient. In some embodiments, administration of the vectors, nucleic acids, or cells disclosed herein increases cell death in a treated patient or in vitro or ex vivo organ or tissue between day 1 and year 10. In some embodiments, administration of the nucleic acids, vectors, or cells disclosed herein reduces metastasis at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients, or in vitro or ex vivo organ or tissue treated with other cell-killing methods. In some embodiments, administration of the nucleic acids, vectors, or cells disclosed herein increases cell death for about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with controls or patients, or in vitro or ex vivo organ or tissue treated with other cell-killing methods.

In some embodiments, cell death increases by about 1%, about 5%, about 10%, about 20%, about 30%, about 40%, about 50%, about 60%, about 70%, about 80%, about 90%, or about 100% compared with controls or patients treated with other cell-killing methods. In some embodiments, administration of the nucleic acids, vectors, or cells disclosed herein increases cell death by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with controls or patients or in vitro or ex vivo organ or tissue treated with other cell-killing methods. In some embodiments, administration of the nucleic acids, vectors, or cells disclosed herein increases cell death by about 1%, about 5%, about 10%, about 20%, about 30%, about 40%, about 50%, about 60%, about 70%, about 80%, about 90%, or about 100% for about 1 day, about 2 days, about 3 days, about 4 days, about 5 days, about 6 days, about day 1, about day 2, about day 3, about day 4, about day 5, about day 6, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared controls or patients or in vitro or ex vivo organ or tissue treated with other cell-killing methods.

In some embodiments, administration of the nucleic acids, vectors, or cells disclosed herein reduce disease or disorder symptoms in a treated patient compared to an untreated patient or the same patient before treatment. In some embodiments, the disease or disorder symptoms are measured in a treated patient between day 1 and year 10. In some embodiments, administration of the nucleic acids, vectors, or cells disclosed herein reduces a disease or disorder symptom at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the disease or disorder symptom in an untreated patient or the same patient before treatment. In some embodiments, administration of the nucleic acids, vectors, or cells disclosed herein reduces a disease or disorder symptom for about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with the disease or disorder symptom in an untreated patient or the same patient before treatment.

In some embodiments, the disease or disorder symptom is reduced by about 1%, about 5%, about 10%, about 20%, about 30%, about 40%, about 50%, about 60%, about 70%, about 80%, about 90%, or about 100% compared with the disease or disorder symptom in an untreated patient or the same patient before treatment. In some embodiments, administration of the nucleic acids, vectors, or cells disclosed herein reduces the disease or disorder symptom by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the disease or disorder symptom in an untreated patient or the same patient before treatment. In some embodiments, administration of the nucleic acids, vectors, or cells disclosed herein reduces the disease or disorder symptom by about 1%, about 5%, about 10%, about 20%, about 30%, about 40%, about 50%, about 60%, about 70%, about 80%, about 90%, or about 100% for about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with the disease or disorder symptom in an untreated patient or the same patient before treatment.

In some embodiments, administration of the vectors or nucleic acids disclosed herein reduce cancer symptoms in a treated patient compared to an untreated patient or the same patient before treatment. In some embodiments, the cancer symptoms are measured in a treated patient between day 1 and year 10. In some embodiments, administration of the nucleic acids, vectors, or cells disclosed herein reduces a cancer symptom at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the cancer symptom in an untreated patient or the same patient before treatment. In some embodiments, administration of the nucleic acids, vectors, or cells disclosed herein reduces an cancer symptom for about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more compared with the cancer symptom in an untreated patient or the same patient before treatment.

In some embodiments, the cancer symptom is reduced by about 1%, about 5%, about 10%, about 20%, about 30%, about 40%, about 50%, about 60%, about 70%, about 80%, about 90%, or about 100% compared with the cancer symptom in an untreated patient or the same patient before treatment. In some embodiments, administration of the nucleic acids, vectors, or cells disclosed herein reduces the cancer symptom by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the cancer symptom in an untreated patient or the same patient before treatment. In some embodiments, administration of the nucleic acids, vectors, or cells disclosed herein reduces the cancer symptom by about 1%, about 5%, about 10%, about 20%, about 30%, about 40%, about 50%, about 60%, about 70%, about 80%, about 90%, or about 100% for about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more compared with the cancer symptom in an untreated patient or the same patient before treatment.

In some embodiments, the present disclosure provides methods to precisely track the cells transfected with the compositions of the disclosure. In some embodiments, these cells are traced to determine the behavior of the cells. In some embodiments, these cells are traced to determine their migration. In some embodiments, these cells are traced to determine their metastasis (or lack thereof). In some embodiments, these cells are traced to determine their response to any drugs administered either in vitro or in vivo. In some embodiments, the cells are also exposed to a therapeutic agent. In some embodiments, the therapeutic agent is a chemotherapeutic agent. In some embodiments the cells are exposed to the therapeutic agent in vitro. In some embodiments, the cells are exposed to the therapeutic agent in vivo.

### Co-Administration

In some embodiments, the patient is subjected to additional therapies. In some embodiments, the patient is treated with a composition of the present disclosure as part of an anti-cancer therapy. In some embodiments, the anti-cancer therapy is selected from, but not limited to, radiation therapy, chemotherapy, immunotherapy, hormone therapy, stem cell transplant, and CAR-T therapy. The anti-cancer therapy may be administered before, at the same time, or after administration of the compositions of the disclosure.

### Kits

The disclosure also provides kits for decreasing, preventing, ameliorating, or delaying cell migration or metastasis. In some embodiments, the kits include a vector or nucleic acid of the present disclosure. The kit can further include a label or printed instructions instructing the use of described reagents. The kit can further include a treatment to be tested. The kits are applied for in vitro and in vivo cell migration or metastasis prevention, decrease, amelioration, or delay.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

“Anti-oncogene” as used herein refers to any gene that prevents, delays, inhibits, or otherwise alters expression or activity of oncogenes. In some embodiments, the anti-oncogene is a tumor suppressing gene. In some embodiments, the anti-oncogene decreases expression of a cancer biomarker associated with metastasis.

It should be understood that singular forms such as “a,” “an,” and “the” are used throughout this application for convenience, however, except where context or an explicit statement indicates otherwise, the singular forms are intended to include the plural. All numerical ranges should be understood to include each and every numerical point within the numerical range, and should be interpreted as reciting each and every numerical point individually. The endpoints of all ranges directed to the same component or property are inclusive, and intended to be independently combinable.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

The term “about”, as used herein to refer to a numerical quantity, includes “exactly” plus or minus up to 10% of that referenced numeric indication. When the term “about” is used in reference to a range of values, the term “about” refers to both the minimum and maximum value of the range (e.g., “about 1-50 μm” means “about 1 μm to about 50 μm”). The term “intimately associated”, as used herein to describe the spatial relationship between two or more components of a composition refers to components that are intimately mixed, such as, for example, in mixtures, coatings and matrices.

The terms “transduction” and “transfection” are used interchangeably herein and refer to the introduction of genetic material into a cell. Any appropriate means of genetic modification is envisaged by the present disclosure.

This disclosure is further illustrated by the following non-limiting examples.

### EXAMPLES

### Example 1—Construction of Vectors Expressing PCBP1 Mutant Polypeptides

Studies have shown that mRNA expression of PRL-3 is elevated in metastatic cancers, including ovarian cancer, gastric cancer, and overexpression of PRL-3 negatively correlates with liver cancer prognosis (Bardelli et al. 2003; Polato et al., 2005; Peng et al. 2004). PCBP1 is known to regulate PRL-3 expression, with an inverse relationship between PCBP1 expression and PRL-3 expression which suggests PCBP1 may repress PRL-3 mRNA translation. The downregulation of PRL-3 is accompanied by the downregulation of the phosphorylated active form of Akt (pSer473) (Wang et al. 2010). For example, cancer cells that show an increase of Akt-2 (pSer474) phosphorylation also show a significant reduction in PCBP1 expression (Brown et al., 2016).

As shown in FIG. 1A and 1B, a lentiviral gene therapy vector (LVV), or/and AAV systems containing a PCBP1 gene respectively were generated. This vector system contains the human elongation factor-1 alpha (EF1a) promoter and an IRES element which provides for internal initiation of protein translation from an mRNA molecule. A dicistronic mRNA is thus made where the IRES motif can initiate the independent and separate translation of a second gene from the single PCBPC-IRES-GFP mRNA. Several PCBP1 mutants were constructed that were designed to decrease phosphorylation of the protein by PAK1 based on the strategy below in Table 2. The PCBP1 mutant nucleotide sequences were cloned into the vector shown in FIG. 1A-B to create Vector A (mPCBP1_1, S223L) and Vector B (mPCBP1_2, T60A & T127A). The mutations in each PCBP1 mutant prevent PCBP1 phosphorylation by PAK1. Turn-on of the different genes is driven by the appropriate promoter(s). As PAK1 phosphorylates PCBP1 at both amino acids 60 and 127, mutation at these sites prevent PCBP1 phosphorylation by PAK1.

TABLE 2
Design of Mutant PCBP1

| | | | | | AA | |
|---|---|---|---|---|---|---|
| | | | | | sequence | |
| Mutation | | Wild | | DNA sequence | (mutation | |
| Site | Position | Type | Mutate to | (mutation region) | region) | Vector |
| 1 | AA 223 | TCG | TTG | gcctac tTg attcaa | AY L IQ | Vector |
| | (Nt. #668) | (Ser) | (Leu) | (SEQ ID NO: 8) | (SEQ ID NO: 11) | 1A/1B |
| 2 | AA60 | ACT | GCT(Ala) | atcatc Gct ctg | II A LT | Vector |
| | (Nt. #178) | (Thr) | | (SEQ ID NO: 9) | (SEQ ID NO: 12) | 1A/1B |
| 3 | AA127 | ACG | GCG | gagagt Gcg ggggcg | ES A GA | Vector |
| | (Nt. #379) | (Thr) | (Ala) | (SEQ ID NO: 10) | (SEQ ID NO: 13) | 1A/1B |

### Example 2—The PCBP1 Mutants are Expressed at Levels Higher than Endogenous Wild Type

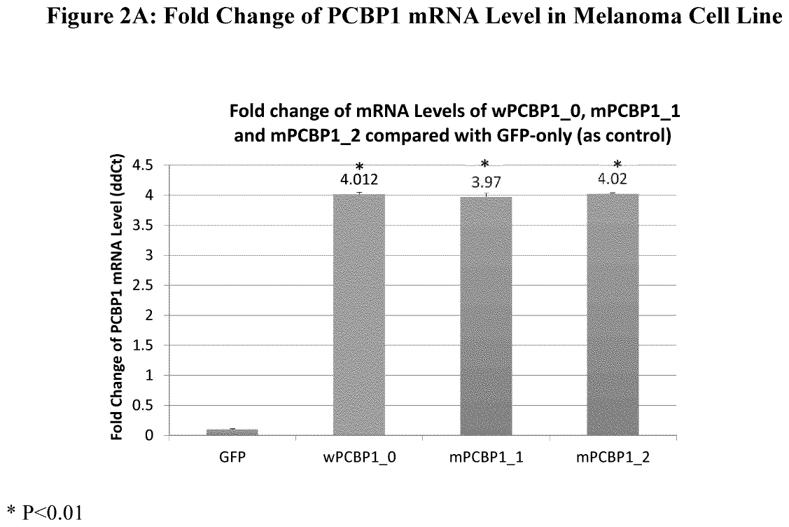

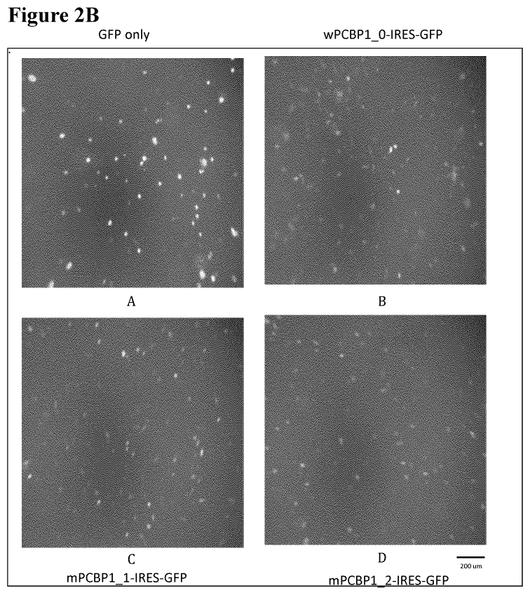

To test whether the PCBP1 S223L and PCBP1 T60A & T127A mutations affected transcription levels, PCBP1 expression in melanoma cells was determined by qRT-PCR. FIG. 2 shows the analysis of fold change between groups (ddct). Comparison of GFP expression with wild type PCBP1 expression (wPCBP1_0) shows PCBP1 expression levels were greater by 4.012 fold. Comparison of GFP expression with expression of PCBP1 S223L (mPCBP1_1) shows PCBP1 S223L expression levels were greater by 3.97 fold. Comparison of GFP expression with expression of PCBP1 S223L mutant shows PCBP1 T60A & T127A (mPCBP1_2) expression levels were greater by 4.02 fold. The wild type PCBP1 and two mutant forms display similar mRNA expression. The housekeeping gene used in this assay is GAPDH.

### Example 3-Expression of PCBP 1 in Melanoma Cells

Melanoma cells were split into four groups and each group was transfected in vitro with vectors containing A) GFP only; B) wild type PCBP1 (wPCBP1_0-IRES-GFP) and GFP; 3) PCBP1 S223L and GFP (mPCBP1_1-IRES-GFP); or D) PCBP1 T60A & T127A (mPCBP1_2-IRES-GFP). 24-48 hours after transfection of lentiviral vectors containing either wild-type PCBP1 or mutant PCBP1 using the lipofectamine kit (Invitrogen Inc.), expression of GFP was observed (FIG. 2). Transfection rates were each around 90% (data not shown). Variation in GFP expression levels may be due to the different vectors. Without being bound by theory, in the vectors, PCBP1 is upstream of the GFP. When PCBP1 efficiency is 100%, the downstream gene (GFP) will show a lower expression efficiency (e.g. around 60%-80%). As the control vector contains only GFP and no PCBP1 sequence, the GFP expression efficiency is higher. There is no evidence indicating that PCBP1 and GFP interact with each other.

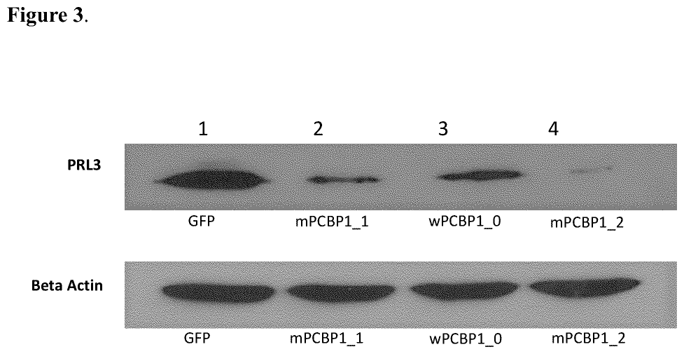

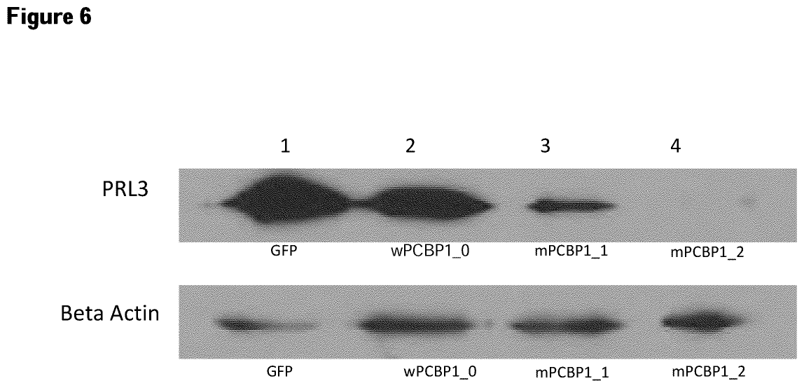

For the Western Blot, briefly after PCPB1 treatment, the cell culture dishes were placed on ice and the cells were washed with ice-cold PBS three times. Ice-cold lysis buffer (containing a protease inhibitor) was then added to the cells. Adherent cells were scraped off the dishes and the cell suspension was transferred into a pre-cooled microcentrifuge tube, which was constantly agitated for 30 minutes at 4° C. and then centrifuged. After centrifugation, the tubes were placed on ice, the supernatant aspirated to a fresh tube, and the pellet was discarded. After protein quantification, the protein concentration of each cell lysate was determined by adding an equal volume of 2×Laemmli sample buffer. The sample was boiled in this sample buffer at 100° C. for 5 minutes. Equal amounts of protein from the treatment and control groups were loaded into each well of an SDS-PAGE gel, along with a molecular weight marker. The protein concentrations tested ranged from about 20-30 μg from the cell lysate or tissue homogenate, or about 10-100 ng of purified protein. The gel was electrophoresed for 1-2 hours at 100V. The proteins on the gel were then transferred to an active nitrocellulose membrane using transfer buffer and the Bio-Rad Semi-Transfer system (Bio-Rad, CA). The membrane was blocked by using the blocking buffer for 1 hour at room temperature or overnight at 4° C., and then washed 3 times (5 min/per each washing) with TBST buffer before starting antibody staining. The membrane was then incubated with the chemiluminescence substrate for about 5 min to visualize the protein signal following the kit manufacturer's recommendation (See FIG. 3 and FIG. 6).

FIG. 3 shows the PRL-3 protein expression in the cells transfected with the gene therapy vectors encoding different forms of PCBP1. As shown in FIG. 3, transfection of the melanoma cell with vector containing wild type PCBP1 decreases PRL-3 expression compared with the GFP-only vector. Transfection of the cells with a vector containing either of the PCBP1 mutants decreases PRL-3 expression even further, with the double PCBP1 T60A & T127A mutant decreasing PRL-3 expression to an even greater degree than transfection with the single PCBP1 S223L mutant.

### Example 4—Transfection with Mutant PCBP1 Decreases Melanoma Cell Migration

The effect of the mutant, de-phosphorylated PCBP1 mutants on cell migration was tested using a cell migration assay. Briefly, melanoma cells transfected with GFP only, wild type PCBP1 (wPCBP1_0-IRES-GFP) and GFP, or PCBP1 T60A & T127A (mPCBP1_2-IRES-GFP) were observed for five days post-transfection.

The same number of cells from each treatment group or/and control was seeded onto 24 (or 6) well-plates for transfection by the vector encoding a marker protein (i.e. GFP) using the lipofectamine kit (Invitrogen Inc). The cells transfected with GFP were then suspended by medium containing trypsin (0.1%), and washed by culture medium. The same amount of the cell numbers (e.g. about 80,000 cells from each individual group) was then re-placed and evenly distributed onto the Oris migration assay plate (Oris Cell Migration Assay kit) with a loading volume of 100 μl containing the same amount of cell numbers into each well of the 96 well-plate. Each well of the dish containing the Oris™ Cell Seeding Stoppers (OCS) was then incubated in a humidified chamber (37° C., 5% CO2) for 5 to 10 hours (cell line dependent) to permit cell attachment. Before counting the cells, the OCS was removed by using an Oris™ Stopper tool to create the areas termed “Circle Detection Zone (CDZ)” for detecting any new cells that migrated to inside this CDZ as new migration cells. Those cells expressing the GFP in the CDZ were then counted at each time-point using the microplate reader (Molecular Devices, LLC) or/and under a fluorescent microscope (BD Biosciences).

Cells were visualized and analyzed by fluorescence microscopy of the GFP expression. The GFP reflects the expression of the genes in each transfected cell, although the intensity of the GFP may decrease the level of intensity along with the time, although as shown in the results, not much fluorescence intensity was lost over 5 days. The total cells displaying intensity for each well was measured at the same time (day) point (e.g. Day 0 or Day 5). As the cells with GFP were transduced by the vector encoding the genes including GFP, and not by immunostaining with a fluorescent dye, the fluorescent signal is not detached from the substrate. Those cells not displaying a fluorescent signal are not transfected with the vectors of the disclosure and are therefore not counted and limited out in this analysis.

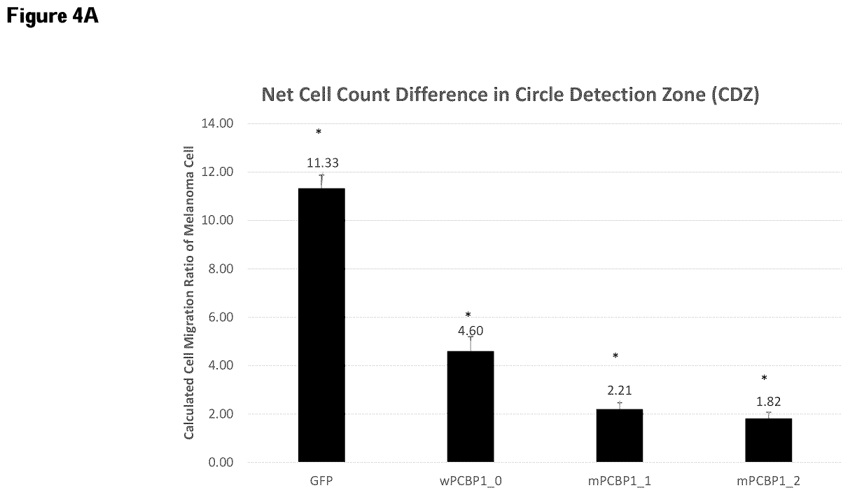

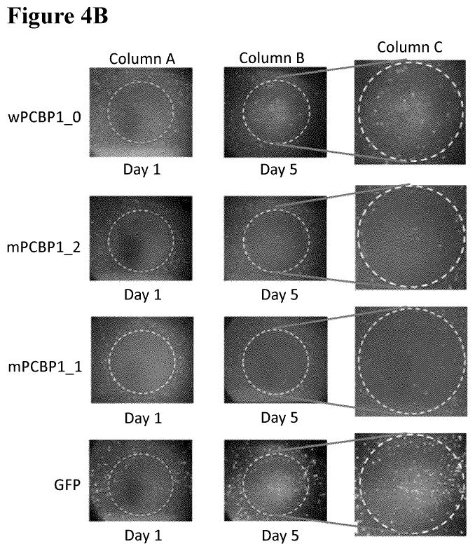

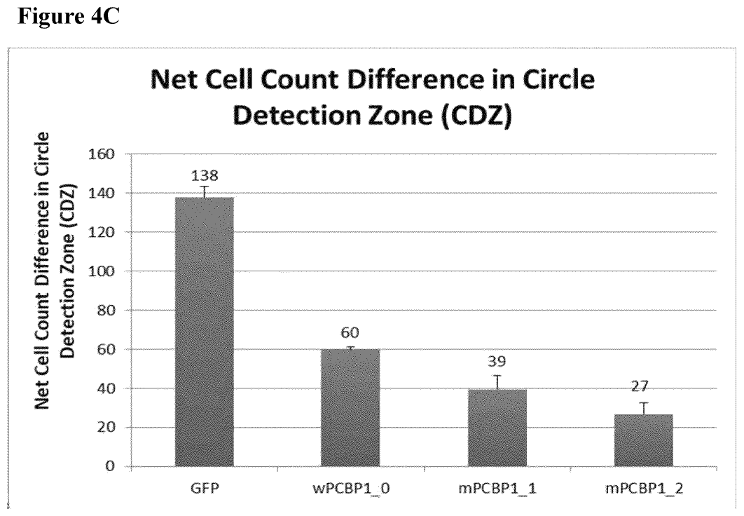

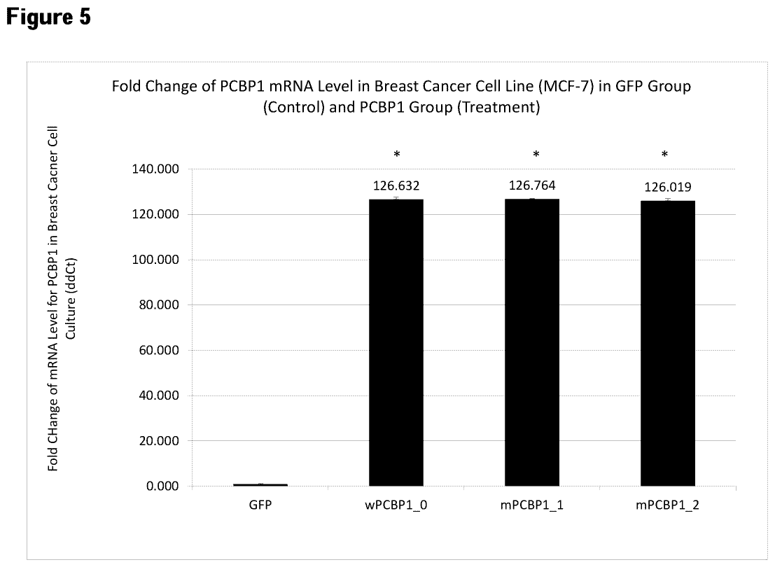

The results of this cell migration assay are demonstrated in FIG. 4A-C. In FIG. 4B, the white dotted line circle is the Circle Detection Zone (CDZ). Column A is Day 1; Column B is cell migration well in day 5 (with the CDZ in a white dotted line); Column C is the enlarged CDZ from Column B. Table 3 shows the quantified data from the cell migration assay which demonstrates the double T60A&T127A mutant exhibited a decreased net cell migration in the CDZ compared with both wild type PCBP1 and the GFP-only cells.

TABLE 3
Calculation of Net Cell
Migration of melanoma cells into CDZ

| | Net Cell | |
|---|---|---|
| Vector | Migration in CDZ | Std |
| GFP | 138 | 7.071 |
| wPCBP1_0 | 60 | 7.778 |
| mPCBP1_1 (S223L) | 39 | 2.828 |
| mPCBP1_2 | 27 | 6.364 |
| (T60A & T127A) | | |

FIGS. 4A-4C show the cell migration adjusted to percentage of cells per well. The percentage of migrated cells (per well) in the GFP group (11.076) was significantly higher than that of wild type PCBP1 (wPCBP1_0) and PCBP1 T60A & T127A (mPCBP1_2). Among these three groups, mPCBP1_2 displayed the lowest percentage of migrated cells per well (2.082). These values were calculated as follows:

In each time point, the total cells were detected by microplate reader (as a base denominator). Cells were also counted in the region of circle detection zone (CDZ) as migrated cells per time point (as the numerator). The adjusted net cell difference in the CDZ was calculated according to the formula shown as below:

$$Percentage of migrated cells per well ( \% ) = \frac{Day 5 of cells counted in the CDZ}{\begin{matrix}Day 5 of total cells signals by microplate \\ Reader per well\end{matrix}} - \frac{Day 0 of cells counted in the CDZ}{\begin{matrix}Day 0 of total cells signals by microplate \\ Reader per well\end{matrix}}$$

### Example 5—Transfection with Mutant PCBP1 Decreases Breast Cancer Cell Migration

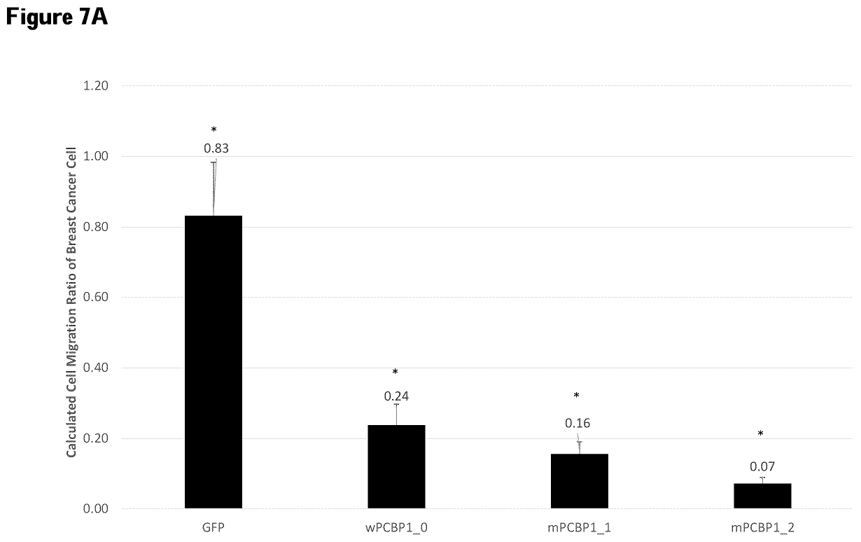

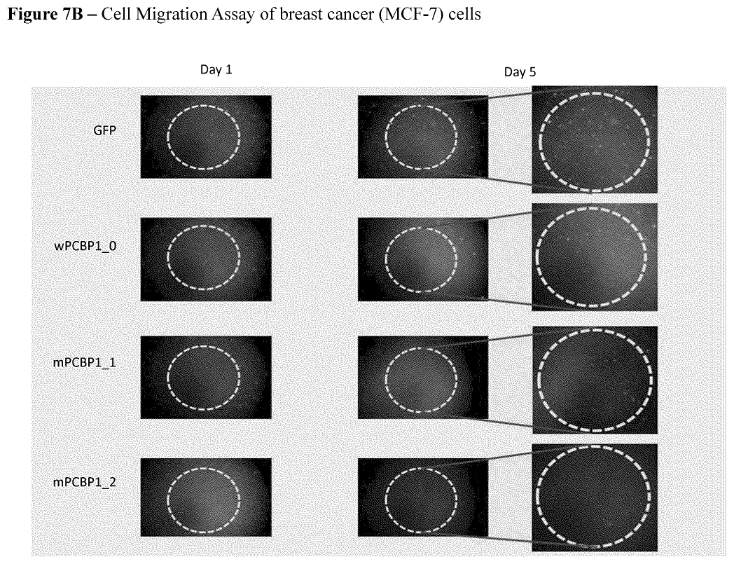

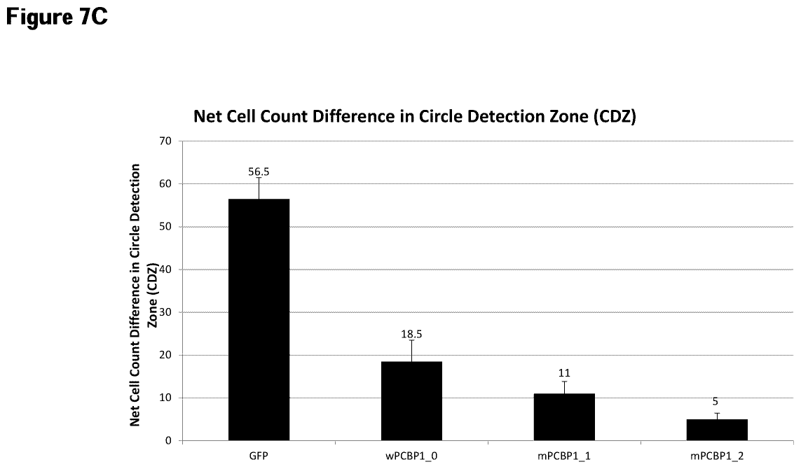

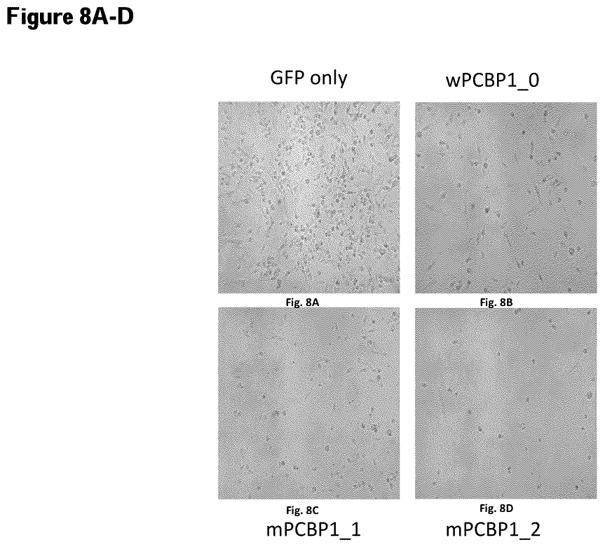

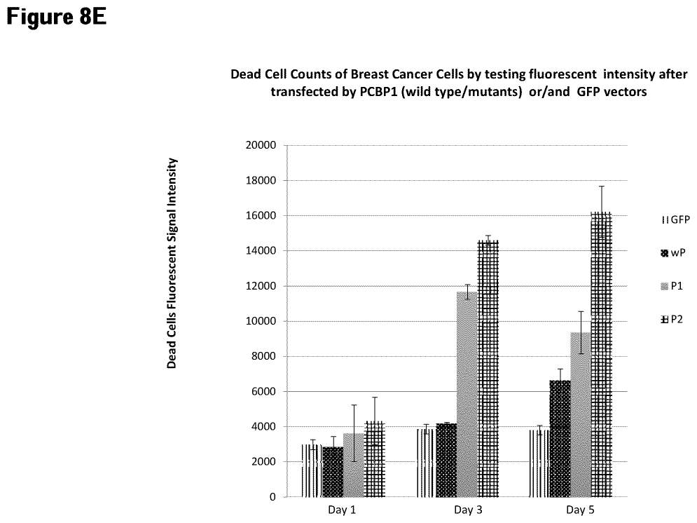

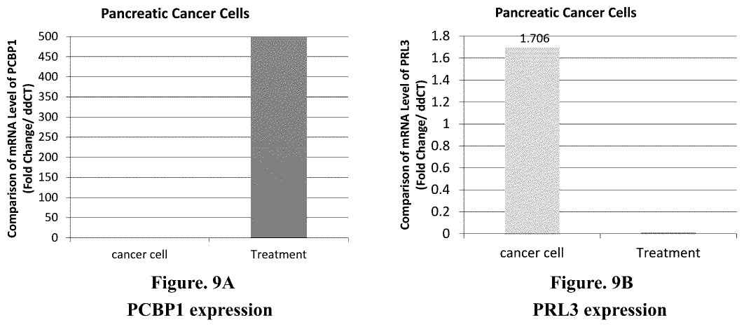

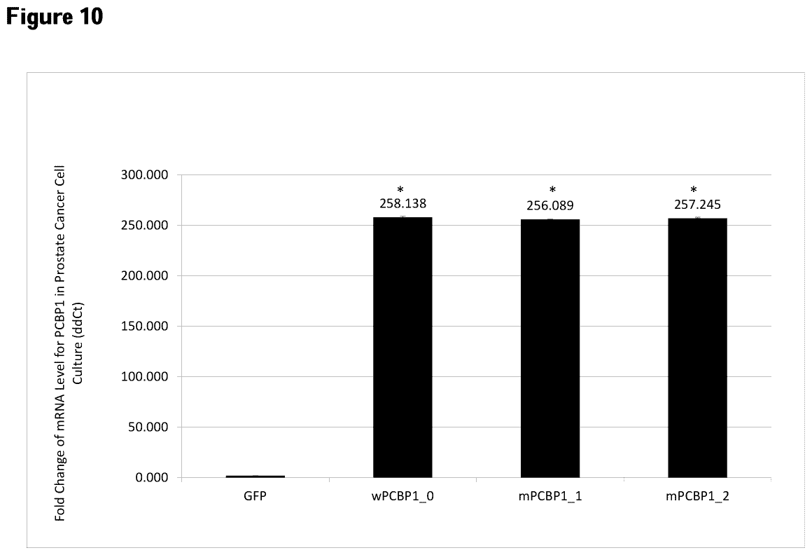

The breast cancer cells from the MCF-7 cell line were transfected with vectors containing GFP only, wild type PCBP1 (wPCBP1_0-IRES-GFP) and GFP, or PCBP1 T60A & T127A (mPCBP1_2-IRES-GFP) and were observed for five days post-transfection. The migration of these cells was observed as detailed above. Table 4 shows the net cell migration in CDZ of the different transfected cells. FIGS. 7A-C show the cell migration adjusted to percentage of cells per well. The percentage of migrated cells (per well) in the GFP group was significantly higher than that of wild type PCBP1 (wPCBP1_0) and PCBP1 T60A & T127A (mPCBP1_2). Among these three groups, mPCBP1_2 displayed the lowest percentage of migrated cells per well.

TABLE 4
Calculation of Net Cell Migration of
melanoma cells into CDZ

| | Net Cell | |
|---|---|---|
| | Migration | |
| Vector | in CDZ | Std |
| GFP | 56.5 | 4.950 |
| wPCBP1_0 | 18.5 | 4.950 |
| mPCBP1_1 (S223L) | 11 | 2.828 |
| mPCBP1_2 | 5 | 1.414 |
| (T60A & T127A) | | |

### Example 6—Use of PCBP 1 Mutants to Inhibit Tumor Metastasis

### Design of the Animal Model to Study Cancer Gene Therapy:

Experimental Design: A cancer cell line (i.e., MCF-7, ATCC for breast cancer model-BC) is tumorigenic in mice and can be used to study breast cancer (BC) tumor growth in immune deficient animals (i.e., Nude mice strain: BALB/c from Charles River Lab, NC). These cells will be used to develop an in vivo precision method for testing therapeutic efficacy. Initially, it will be prepared for injectable cancer cell lines that are stably transduced by the vector with a marked gene, i.e., encoding red fluorescent protein (RFP). This will allow monitoring growth and migration of the cancer cells, illustrated or monitored by the fluorescent bio-imaging system or fluorescent microscope. After the cancer cells are prepared, they will be injected subcutaneously into mice to generate breast cancer tumors after a week. By using this new method, cancer cells will be precisely traced and studied during their metastasis, following treatment of gene therapeutic vector such as AAV-PCBP1m.

Procedures will be performed as follows with an example of a BC study:
(1) The RFP will be delivered into a BC cell line (MCF-7, ATCC), such as by using a lentiviral-backbone for integration of the RFP gene into the cell genome for stable and long-term expression of the RFP reporter gene. After confirming successful RFP transduction into the cells (by using a fluorescent microscope or FACS), BC cells will be selected and injected.
(2) Mice will be divided into four groups (n=6). RFP-BC cells will be injected into the flank of mice at same conditions (same cell count, injection time) in all groups. BC tumor sizes will be assessed and recorded daily or twice every week. Experimental design and expected outcome will be as described in the following Table 5:

TABLE 5
Example of the In vivo Experiment
Designed with Implantation of BC in Mice.

| Grouping | | | |
|---|---|---|---|
| Group A (GA) | | Group B (GB) | |
| Transduced BC cells with RFT | | | |
| RFP-BC | | RFP-BC | |
| Sub-group | | | |
| GA-1 | GA-2 | GB-1 | GB-2 |

Injection of therapeutic vectors (Day 7)

| | | | | |
|---|---|---|---|---|
| Treatment | PCBP1 | PCBP1 (2 site | Control | Control (no |
| | (WT)- | mutation)- | (empty | vector) |
| | GFP | GFP | vector) | |
| Expectation | Tumor | Tumor | Tumor | Tumor |
| of tumor | suppressed | strongly | grow | grow |
| growth | | suppressed | fast | fast |

The vector containing therapeutic genes will be injected into the tumors directly. Mice will be sacrificed 3 weeks after treatment, for histology studies. Tumor tissues will be extracted for immunohistochemistry (IHC) assay for RFP, GFP, PRL-3, PCBP1 and SOX2 (a BC cell marker). RFP and GFP will be detected on these tissues to confirm BC cell growth and vector delivery, as described in Table above. Additional tissues (including lung, liver, brain and pancreas) will be extracted for tumorigenicity and metastasis studies. RFP and GFP will be detected in these tissues to monitor if BC migrated into these tissues. qRT-PCR, WB and IHC will be also performed. Proteomics studies will be done by two-dimensional gel electrophoresis combined with mass spectrometry techniques to compare protein expression in mice with control vs. PCBP1 mutant transduction. The difference among experimental groups will be compared using student's t test. Statistical significance will be determined as P<0.05.
(3) The analysis of the outcome for the results after treatment may be expected as following Table 6:

TABLE 6
In vitro experimental designed and expected results

| | | Transfected | Transfected |
|---|---|---|---|
| | | by AAV- | by AAV- |
| | Transfected | PCBP1w- | PCBP1m |
| Groups | by | GFP | (different |
| (i.e., breast cancer | AAV-GFP | (wild-type) | new mutations)- |
| cells) | vector | vector | GFP |
| Green Fluorescent Signal | Yes | Yes | Yes |
| The mRNA levels of PCBP1 | + | +++ | +++ |
| determined by qRT-PCR | | | |
| The protein levels of PCBP1 | − | +++ | +++ |
| determined by WB | | | |
| The mRNA levels of PRL3 | +++ | ++ | ++ |
| determined by qRT-PCR | | | |
| Protein levels of PRL3 | ++++ | ++ | +/− |
| determined by WB | | | |
| Cancer cell migration | | | |
| determined by migration | ++++ | ++ | +/− |
| assay | | | |
| Cancer cell apoptosis assay | − | ++ | ++++ |
| Cell Proliferation Assay | ++++ | ++ | +/− |

### Example 7—Treatment of Glioblastoma by Expressing PCBP1 in the Tumor Cells

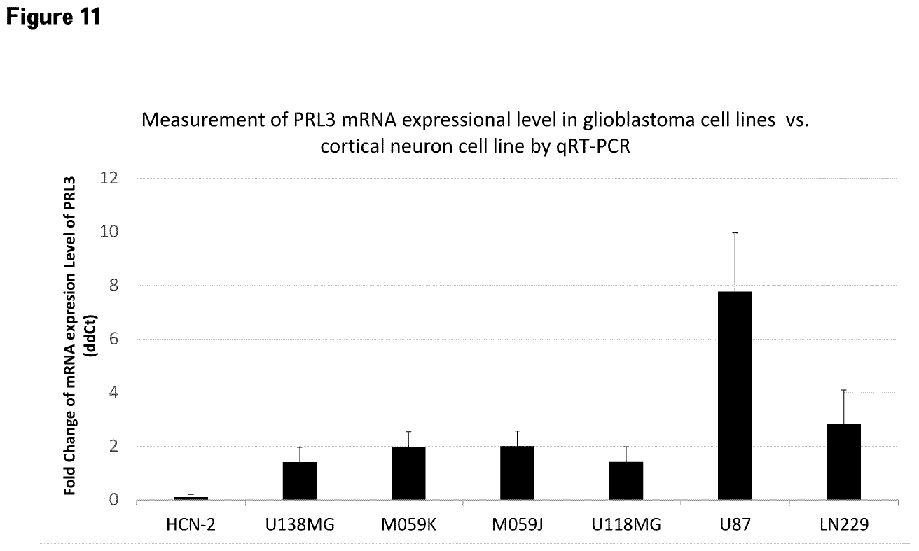

Elevated levels of PRL3 mRNA are found in glioblastoma cells. As shown in FIG. 11, compared to expression in a normal brain cortical cell line, elevated PRL-3 expression was detected in six different glioblastoma cell lines (U138MG, M059K, M059J, U118MG, U87, and LN229). The cell lines U87 and LN229 show the greatest increase in PRL-3 mRNA expression and were selected for further testing.

### Overexpression of Wild Type or Mutant PCBP1 Decreases PRL3 Expression in Glioblastoma Cells

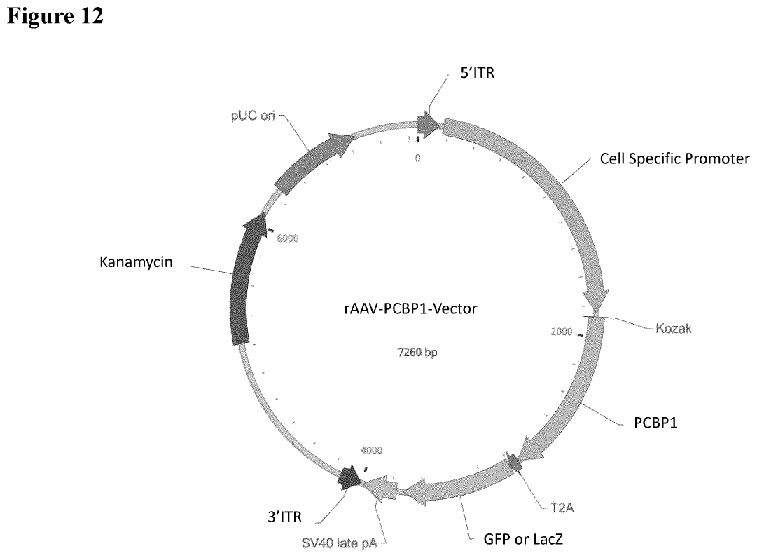

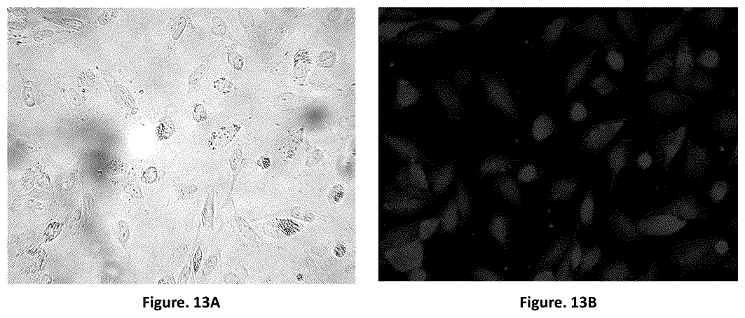

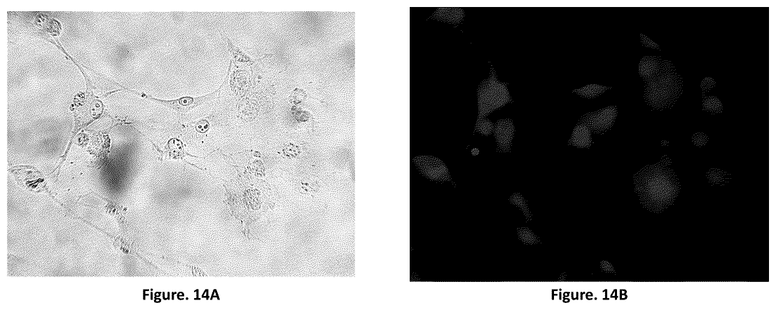

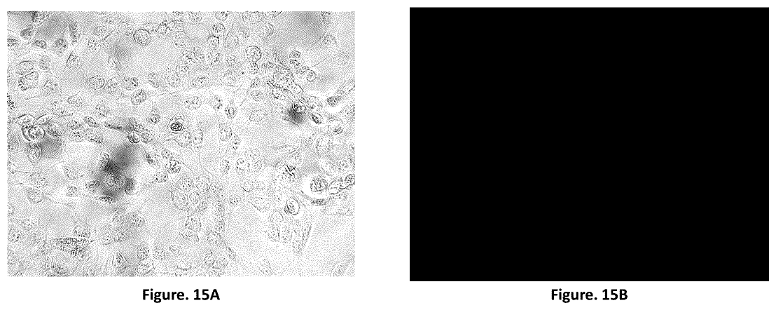

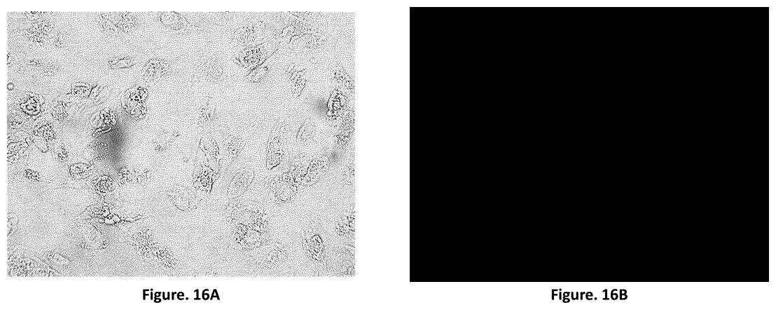

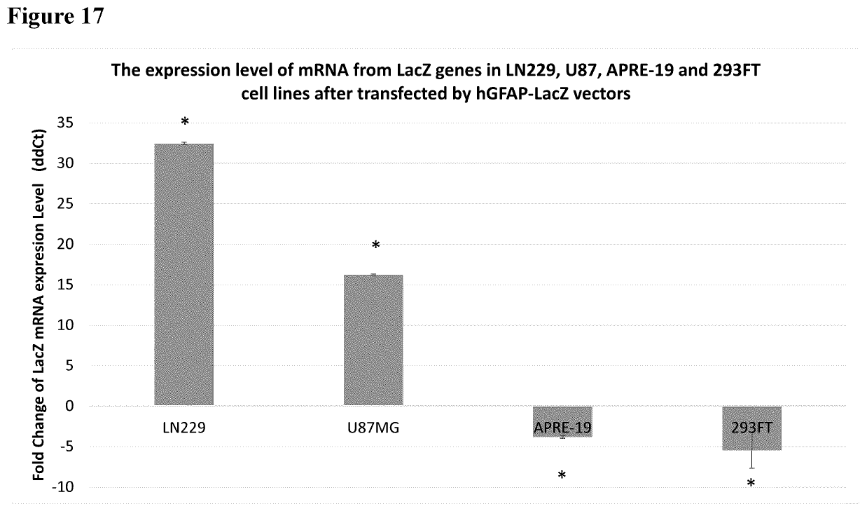

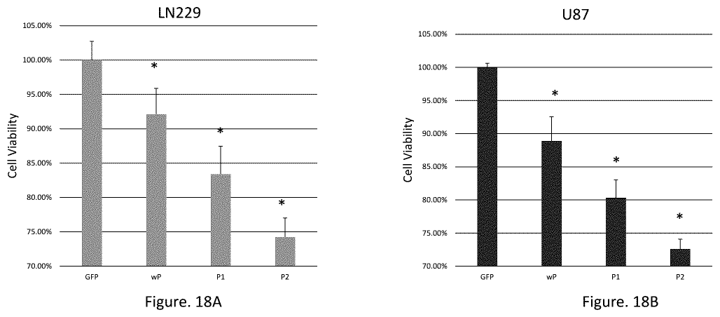

As previously demonstrated, expression of wild type or mutant PCBP1 decreased expression of a gene associated with metastasis, PRL-3, and inhibited cell migration. To test the effect of overexpression of PCBP1 sequences, six different glioblastoma cell lines were transfected with rAAV9 expression vectors containing a wild type or mutant PCBP1 sequence of the disclosure under control of a glioblastoma cell-specific promoter, glial fibrillary acidic protein (GFAP) that enables expression of AP-PCBP1 in glioblastoma cells (see FIG. 12). FIGS. 13-16 demonstrate the specificity of this vector expression in glioblastoma cells. FIG. 13 shows immunohistochemistry analysis of LN299 glioblastoma cells treated with the phGFAP-LacZ vector. FIG. 14 shows immunohistochemistry analysis of LN299 glioblastoma cells treated with the phGFAP-LacZ vector. As shown in FIGS. 13B and 14B, the LacZ gene driven by the GFAP promoter is detected by an anti-β-galactosidase specific antibody, A11132 (ThermoFisher at a dilution of 1:700) in both transfected glioblastoma cell lines. However, β-galactosidase is not detected in kidney (FIG. 15) or eye (FIG. 16) cells indicating this vector will express its cargo only in glioblastoma cells. A comparison of the LacZ mRNA expression levels is shown in FIG. 17.

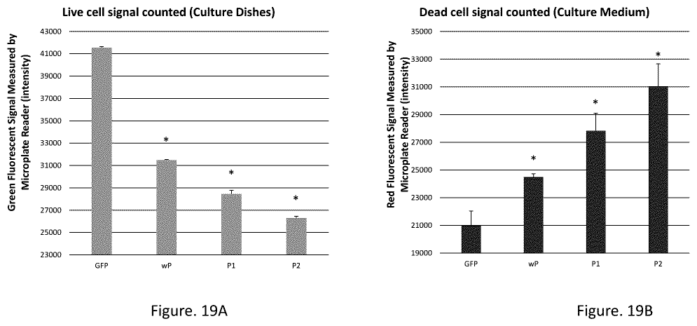

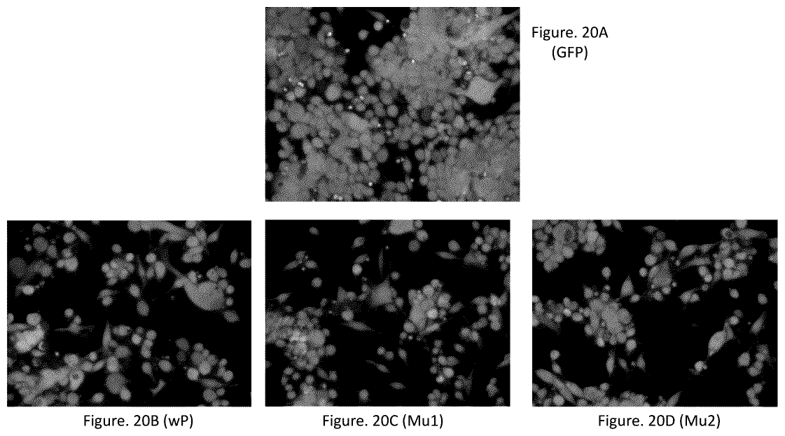

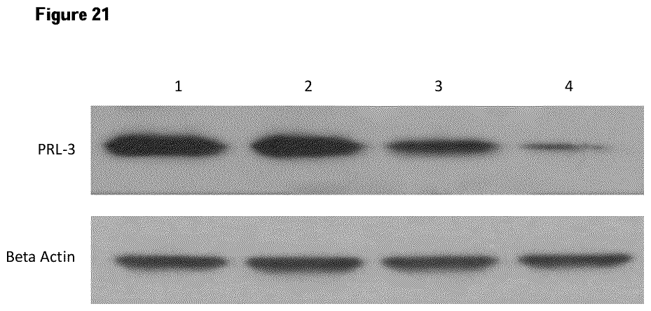

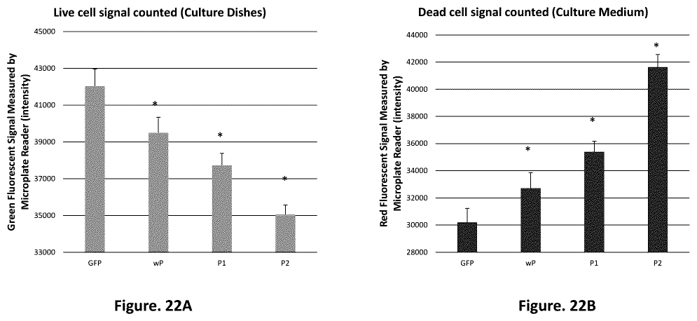

Two micrograms of an AAV vector containing 1) GFP only; 2) wild type PCBP1; 3) mPCBP1_1; or 4) mPCBP1_2 was transfected into the LN229 and U87 glioblastoma cell lines and cell proliferation was tested by using the Vybrant MTT kit. As shown in FIGS. 18A-B, overexpression of wild type PCBP1 or either mutant significantly inhibit the proliferation of the glioblastoma cells when compared with non-treatment with the AAV-GFP vector control. Further, overexpression of a PCBP1 mRNA increases the number of dead cells in the culture, as shown in FIGS. 19 and 20. Dead and live U87 glioblastoma cells were analyzed using the LIVE/DEAD™ Viability Kit (Thermofisher, Inc.) after transfection by AAV-GFP and AAV-PCBP1 and mutants. FIG. 19A shows that the control-treated cell culture contains significantly more live cells than cells transfected with vectors containing wild type or mutant PCPB1 sequences, and FIG. 19B shows that these PCBP1 cell cultures contain significantly more dead cells than the control-treated cell culture. FIGS. 20A-D show the staining of live/dead U87 cells after transfection with either a GFP-control vector or a vector containing a PCBP1 sequence. As shown in FIG. 21, treatment by all forms of PCBP1 downregulated PRL3 protein expression in the U87 glioblastoma cell line, with the greatest decrease in PRL3 expression observed in cells treated with mPCBP1_2.

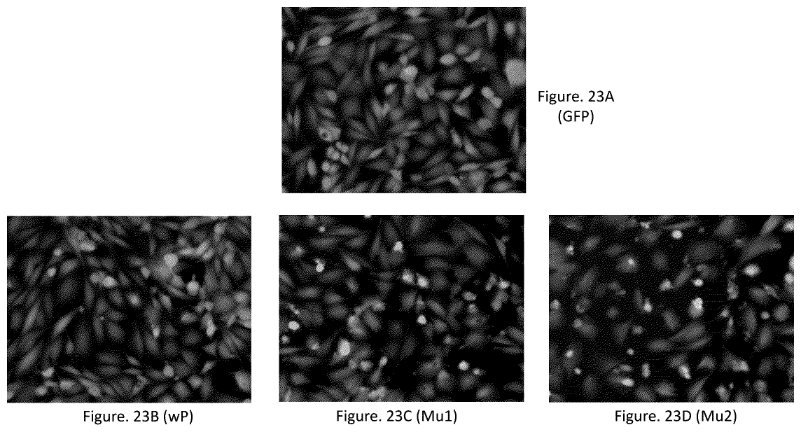

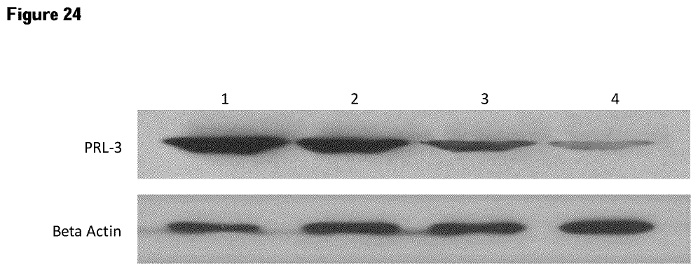

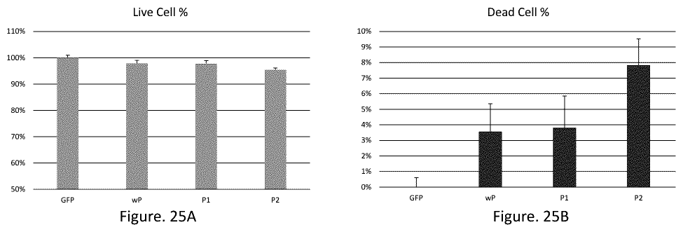

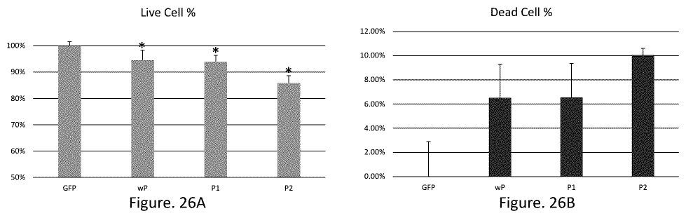

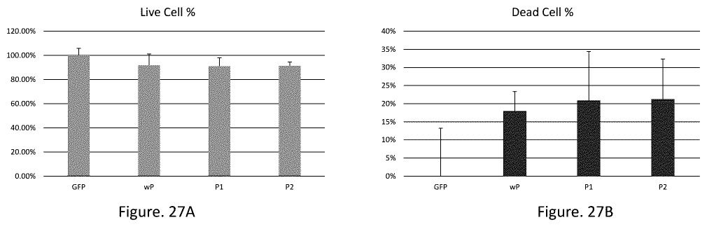

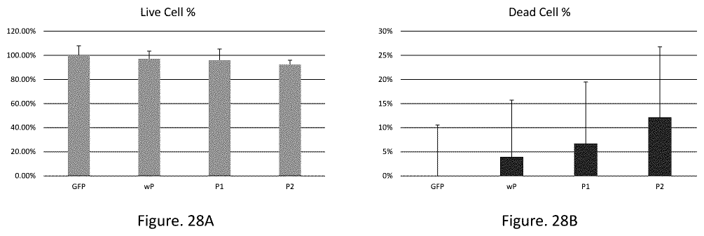

These results were not specific to the U87 cell line, and were also demonstrated in other glioblastoma cell lines. Transfection of LN299 glioblastoma cells with wild type or mutant PCBP1 increased the number of dead cells in the cell culture (FIGS. 22 and 23), and decreased levels of PRL3 protein expression (FIG. 24). Again, treatment with mPCBP1_2 showed the greatest decrease in PRL3 protein expression (FIG. 24). The same effect on cell viability was observed in the M059J (FIG. 25), M059K (FIG. 26), U118MG (FIGS. 27), and U138MG (FIG. 28) glioblastoma cell lines.

### Overexpression of Wild Type or Mutant PCBP1 Decreases STAT3 Expression in Glioblastoma Cells

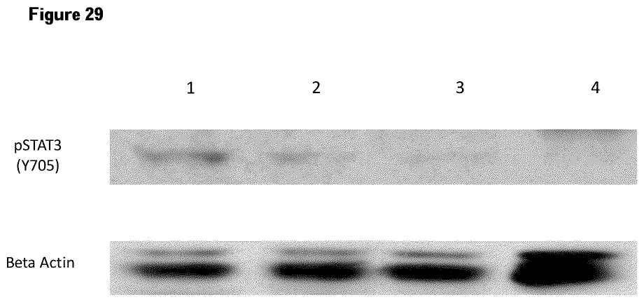

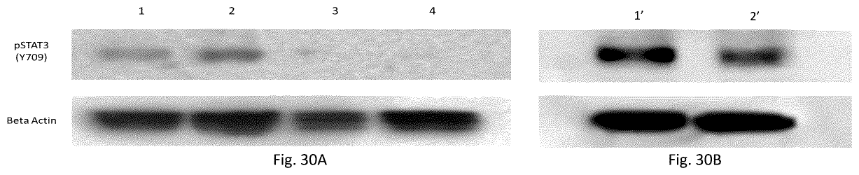

Like PRL3, the expression of STAT3, a marker of cancer, was decreased in glioblastoma cells transfected with vectors containing wild type or mutant PCBP1 sequences. FIGS. 29 and 30 show STAT3 protein expression levels are decreased in U87 and LN299 glioblastoma cells transfected with wild type PCBP1, mPCBP1_1, or mPCBP1_2, with the mPCBP1_2 treated cells demonstrating the greatest decrease in STAT3 protein levels.

### Example 8—Treatment of Ovarian and Prostate Cancer by Expressing PCBP1 in the Tumor Cells

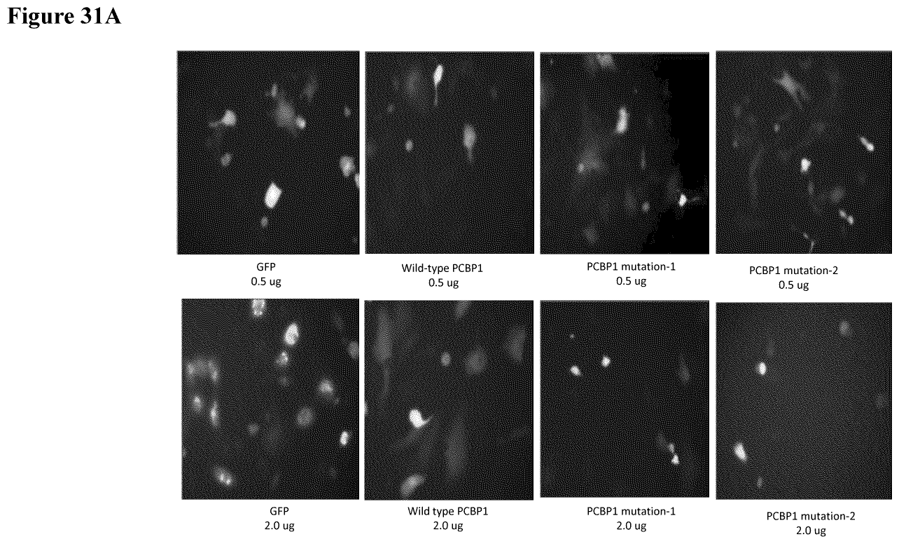

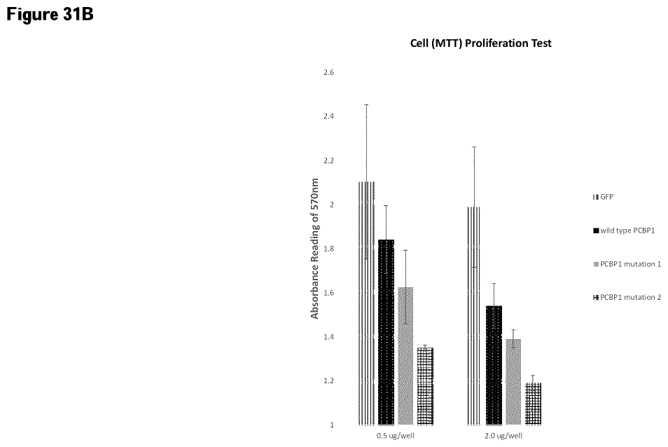

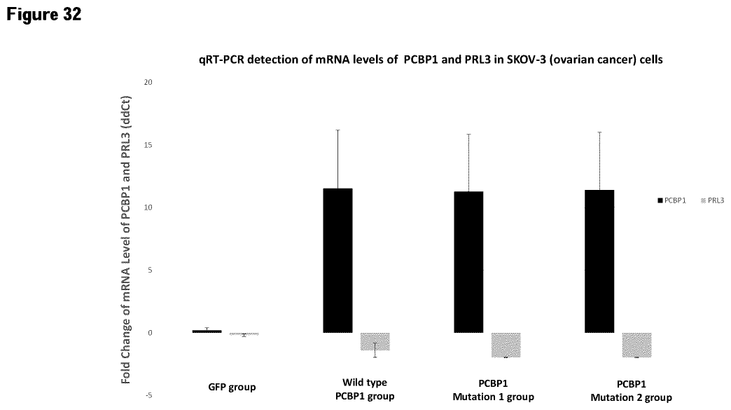

To demonstrate the effect of PCBP1 overexpression is not restricted to glioblastoma cells, the SKOV3 ovarian cancer cell line was transfected with vectors containing wild type or mutant PCBP1 sequences. SKOV3 cells were transfected with either 0.5 μg or 2.0 μg lentivectors containing GFP, PCBP1, mPCBP1_1, or mPCBP1_2. Cell proliferation was assayed using MTT (Thermofisher Inc.). As shown in FIG. 31, transfection of all three PCBP1-containing vectors increased inhibition of cell proliferation in a dose-dependent manner. Further, FIG. 32 demonstrates transfection with a wild type or mutant PCBP1 sequence decreases PRL3 mRNA expression.

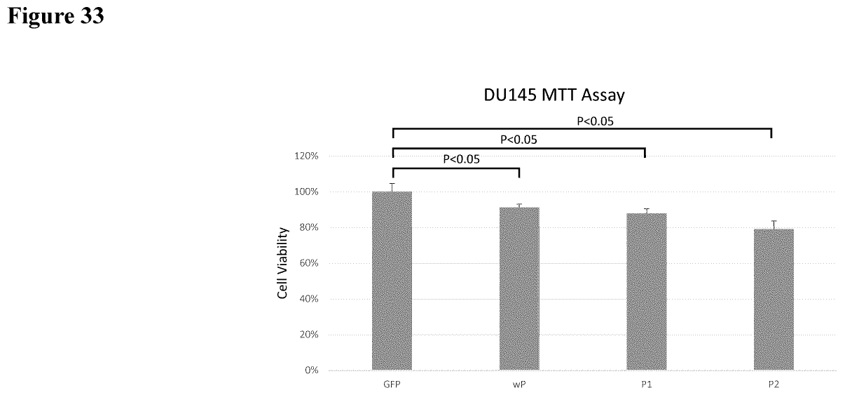

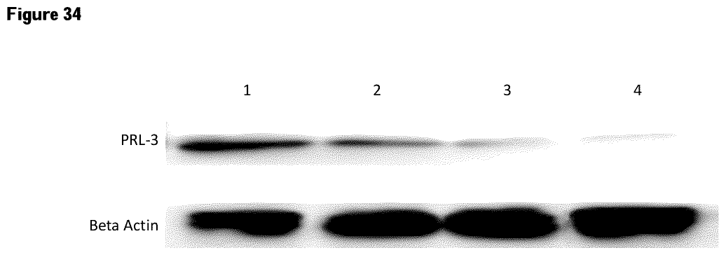

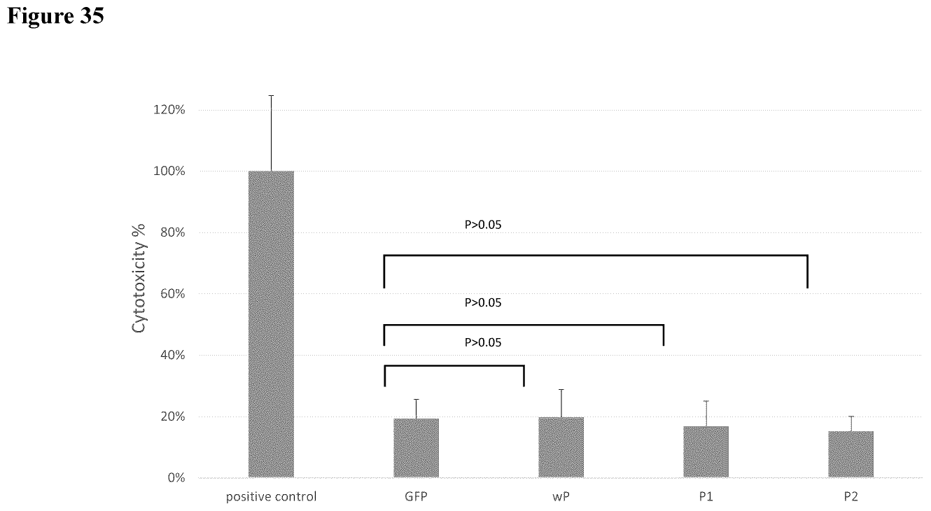

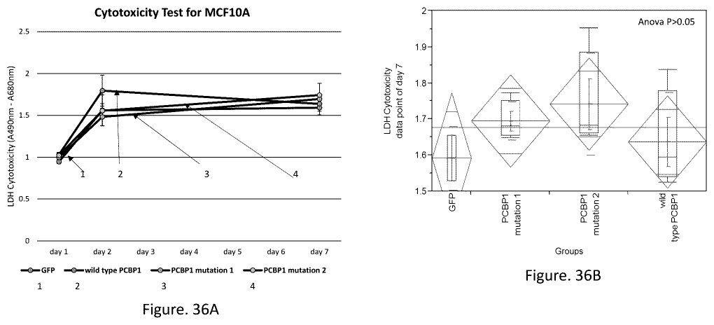

Similar results were observed in DU145 prostate cancer cells transfected with vectors containing wild type or mutant PCBP1 sequences. FIG. 33 demonstrates cells overexpressing PCBP1 showed a significant decrease in cell proliferation when compared to control cells. FIG. 34 shows that PRL3 protein expression levels are similarly decreased, with the mPCBP1_2 mutant showing the greatest decrease.

### Example 9—Overexpression of Wild Type or Mutant PCBP1 is Not Toxic to Non-Cancerous Cells

To demonstrate the overexpression of PCBP1 is not toxic to non-cancerous cells, two normal cell types were transfected with vectors containing wild type or mutant PCBP1 and lactate dehydrogenase (LH) assays were performed. Lactate dehydrogenase is released from damaged cells into the media, and increased levels of LH are thus a biomarker for cytotoxicity and cytolysis. Normal brain cells (HCN-2) and normal breast cells (MCF10A) were each transfected with vectors containing 1) GFP only (e.g. no PCBP1 treatment), 2) wild type PCBP1; 3) mPCBP1_1; and 4) mPCBP1_2. FIGS. 33-34 demonstrate no significant killing of normal brain and breast cells by treatment with PCBP1.

### INCORPORATION BY REFERENCE

All references, applications, publications, patents, and patent publications cited are incorporated by reference herein in their entirety for all purposes.

### REFERENCES

1. J. Krause William (2005-07-01). Krause's Essential Human Histology for Medical Students. Boca Raton: Universal Publishers. ISBN 9781581124682.
2. https://hive.biochemistry.gwu.edu/cgibin/prd/biomuta/servlet.cgi?gpageid=11&searchfield1=gene_name&searchvalue1=PCBP1).
3. Ghanem, L. R. et al. The Poly (C) binding protein Pcbp2, and its retrotransposed derivative Pcbp1, are independently essential to mouse development. Mol. Cell. Biol. 36, MCB.00936-15 (2015).
4. Basak, S. et al. The metastasis-associated gene Prl-3 is a p53 target involved in cell-cycle regulation. Mol. Cell 30, 303-14 (2008).
5. Lai, Z., Han, I., Zirzow, G., Brady, RO and Reiser, J. Intercellular protein delivery by lentivirus vectors using a herpes simplex virus vp22 fusion protein Proc. Natl. Acad. Sci. USA. 2000; 97: 11297-11302.
6. Chaudhury A, Chander P, Howe PH (2010) Heterogeneous nuclear ribonucleoproteins (hnRNPs) in cellular processes: Focus on hnRNP E1's multifunctional regulatory roles. RNA 16 (8): 1449-1462.
7. Lai Z., 2002: Design of an HIV-1 Lentiviral-Based Gene-Trap Vector to Detect Developmentally Regulated Genes in Mammalian Cells. Proceedings of the National Academy of Sciences of the United States of America Vol. 99, No. 6 (Mar. 19, 2002), pp. 3651-3656.
8. Wang H. et al. PCBP1 suppresses the translation of metastasis-associated PRL-3 phosphatase. Cancer Cell. 2010 Jul. 13;18 (1): 52-62. doi: 10.1016/j.ccr.2010.04.028.
9. Reiser, J., Lai, Z., Zhang, X. Y. & Brady, R. O. Development of multigene and regulated lentivirus vectors. J. Virol. 74, 10589-99 (2000).
10. Lai, Z. & Brady, R. O. Gene transfer into the central nervous system in vivo using a recombinanat lentivirus vector. J. Neurosci. Res. 67, 363-371 (2002).
11. Lai, Z., Han, I., Park, M. & Brady, R. O. Design of an HIV-1 lentiviral-based gene-trap vector to detect developmentally regulated genes in mammalian cells. Proc. Natl. Acad. Sci. U.S.A. 99, 3651-6 (2002).
12. Bardelli, S. Saha, J.A. Sager, K.E. Romans, B. Xin, S.D. Markowitz, C. Lengauer, V.E. Velculescu, K.W.Kinzler, B. Vogelstein (2003), PRL-3 expression in metastatic cancers Clin. Cancer Res., 9 pp. 5607-5615
13. F. Polato, A. Codegoni, R. Fruscio, P. Perego, C.Mangioni, S. Saha, A. Bardelli, M. Broggini (2005), PRL-3 phosphatase is implicated in ovarian cancer growth Clin. Cancer Res., 11 pp. 6835-6839
14. L. Peng, J. Ning, L. Meng, C. Shou (2004), The association of the expression level of protein tyrosine phosphatase PRL-3 protein with liver metastasis and prognosis of patients with colorectal cancer J. Cancer Res. Clin. Oncol., 130 pp. 521-526
15. Wang, Haihe et al. (2010) PCBP1 Suppresses the Translation of Metastasis-Associated PRL-3 Phosphatase. Cancer Cell, Volume 18, Issue 1, 52-62
16. PCBP1 mutation database: https://hive.biochemistry.gwu.edu/cgi-bin/prd/biomuta/servlet.cgi?gpageid=11&searchfield1-gene_name&searchvalue1=PCBP1.
17. Qingchang Meng, et al. Signaling-dependent and coordinated regulation of transcription, splicing, and translation resides in a single co-regulator, PCBP1. Proceedings of the National Academy of Sciences. 5866-5871, doi: 10.1073/pnas.0701065104
18. Brown, A (2016). Identification and characterization of an hnRNP E1 translational silencing motif. Nucleic Acids Research. P5892-5907. 2016, Vol. 44, No. 12. doi: 10.1093/nar/gkw241.

1.-33 (canceled)

34. A vector, comprising a sequence encoding a variant or mutant PCBP1 polypeptide comprising one or more variations or mutations selected from the group consisting of: S223L, T60A, and T127A, relative to the wild type PCBP1 polypeptide of SEQ ID NO: 2.

35. The vector of claim 34, wherein the sequence encoding the variant or mutant PCBP1 polypeptide comprises C668T relative to the wild type PCBP1 nucleotide of SEQ ID NO: 1.

36. The vector of claim 34, wherein the sequence encoding the variant or mutant PCBP1 polypeptide comprises A178G and A379G relative to the wild type PCBP1 nucleotide of SEQ ID NO: 1.

37. The vector of claim 35, wherein the variation or mutation is S223L.

38. The vector of claim 36, wherein the variation or mutation is T60A and T127A.

39. The vector of claim 35, wherein the sequence encoding the variant or mutant PCBP1 polypeptide comprising the C668T comprises SEQ ID NO: 3.

40. The vector of claim 36, wherein the sequence encoding the variant or mutant PCBP1 polypeptide comprising the A178G and A379G comprises SEQ ID NO: 5.

41. The vector of claim 34, wherein the vector is a viral vector selected from the group consisting of: lentiviral, retroviral, adenoviral, adeno-associated, replication-competent, vaccinia, and herpes simplex.

42. The vector of claim 41, wherein the vector is the lentiviral vector.

43. The vector of claim 41, wherein the vector is the adeno-associated viral (AAV) vector.

44. The vector of claim 34, wherein the vector is a non-viral vector.

45. The vector of claim 44, wherein the non-viral vector is selected from the group consisting of: naked DNA, plasmid DNA, naked RNA, eukaryotic, and bacterial.

46. The vector of claim 45, wherein the non-viral vector is the naked DNA.

47. A transformed cell comprising the vector of claim 34.

48. The transformed cell of claim 47, wherein the cell is a cancer cell.

49. The transformed cell of claim 48, wherein the cancer cell is selected from the group consisting of: acute lymphoblastic leukemia (ALL), acute myeloid leukemia (AML), adrenocortical carcinoma, anal cancer, appendix cancer, astrocytoma, basal-cell carcinoma, bile duct cancer, bladder cancer, osteosarcoma, malignant fibrous histiocytoma, brainstem glioma, brain cancer, glioblastoma, cerebellar astrocytoma, cerebral glioma, ependymoma, medulloblastoma, supratentorial primitive neuroectodermal cancer, hypothalamic glioma, breast cancer, bronchial adenomas/carcinoids, Burkitt's lymphoma, carcinoid cancer, central nervous system lymphoma, cervical cancer, chronic lymphocytic leukemia (CLL), chronic myelogenous leukemia (CML), chronic myeloproliferative disorder, colon cancer, cutaneous t-cell lymphoma, desmoplastic small round cell tumor, endometrial cancer, ependymoma, esophageal cancer, Ewing's sarcoma, extracranial germ cell cancer, extragonadal germ cell cancer, extrahepatic bile duct cancer, eye cancer, gallbladder cancer, gastric cancer, gastrointestinal stromal tumor (GIST), germ cell cancer, gestational trophoblastic tumor, head and neck cancer, heart cancer, hypopharyngeal cancer, intraocular melanoma, islet cell carcinoma, kidney cancer, laryngeal cancer, hairy cell leukemia, lip and oral cavity cancer, liposarcoma, liver cancer, lung cancer, lymphoma, medulloblastoma, melanoma, Merkel cell carcinoma, mesothelioma, metastatic squamous neck cancer, multiple endocrine neoplasia syndrome, multiple myeloma, mycosis fungoides, myelodysplastic syndrome, myelogenous leukemia, myeloid leukemia, myeloproliferative disorders, nasopharyngeal carcinoma, neuroblastoma, non-Hodgkin lymphoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal cancer, pituitary adenoma, pleuropulmonary blastoma, primary central nervous system lymphoma, prostate cancer, rectal cancer, renal pelvis and ureter cancer, retinoblastoma, rhabdomyosarcoma, salivary gland cancer, sarcoma, Sézary syndrome, skin cancer, small cell lung cancer, small intestine cancer, soft tissue sarcoma, squamous cell carcinoma, squamous neck cancer, stomach cancer, supratentorial primitive neuroectodermal tumor, t-cell lymphoma, testicular cancer, throat cancer, thymoma and thymic carcinoma, thyroid cancer, trophoblastic tumors, urethral cancer, uterine cancer, uterine sarcoma, vaginal cancer, visual pathway and hypothalamic glioma, vulvar cancer, Waldenström macroglobulinemia, and Wilms tumor.

50. The transformed cell of claim 47, wherein the cell is a non-cancerous cell.

51. A transformed cell, comprising: a vector that comprises a sequence encoding a variant or mutant PCBP1 polypeptide comprising one or more variations or mutations selected from the group consisting of: S223L, T60A, and T127A, relative to the wild type PCBP1 polypeptide of SEQ ID NO: 2.

52. The transformed cell of claim 51, wherein the cell expresses PRL-3.

53. The transformed cell of claim 51, wherein the cell is a cancer cell.

54. The transformed cell of claim 53, wherein the cancer cell is a tumor cell.

55. The transformed cell of claim 53, wherein the cancer cell is selected from the group consisting of: acute lymphoblastic leukemia (ALL), acute myeloid leukemia (AML), adrenocortical carcinoma, anal cancer, appendix cancer, astrocytoma, basal-cell carcinoma, bile duct cancer, bladder cancer, osteosarcoma, malignant fibrous histiocytoma, brainstem glioma, brain cancer, glioblastoma, cerebellar astrocytoma, cerebral glioma, ependymoma, medulloblastoma, supratentorial primitive neuroectodermal cancer, hypothalamic glioma, breast cancer, bronchial adenomas/carcinoids, Burkitt's lymphoma, carcinoid cancer, central nervous system lymphoma, cervical cancer, chronic lymphocytic leukemia (CLL), chronic myelogenous leukemia (CML), chronic myeloproliferative disorder, colon cancer, cutaneous t-cell lymphoma, desmoplastic small round cell tumor, endometrial cancer, ependymoma, esophageal cancer, Ewing's sarcoma, extracranial germ cell cancer, extragonadal germ cell cancer, extrahepatic bile duct cancer, eye cancer, gallbladder cancer, gastric (stomach) cancer, gastrointestinal stromal tumor (GIST), germ cell cancer, gestational trophoblastic tumor, head and neck cancer, heart cancer, hypopharyngeal cancer, intraocular melanoma, islet cell carcinoma, kidney cancer, laryngeal cancer, hairy cell leukemia, lip and oral cavity cancer, liposarcoma, liver cancer, lung cancer (e.g. non-small cell, small cell), lymphoma (e.g. AIDS-related, Burkitt, cutaneous T-cell Hodgkin, non-Hodgkin, primary central nervous system), medulloblastoma, melanoma, Merkel cell carcinoma, mesothelioma, metastatic squamous neck cancer, multiple endocrine neoplasia syndrome, multiple myeloma, mycosis fungoides, myelodysplastic syndrome, myelogenous leukemia, myeloid leukemia, myeloproliferative disorders, nasopharyngeal carcinoma, neuroblastoma, non-Hodgkin lymphoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal cancer, pituitary adenoma, pleuropulmonary blastoma, primary central nervous system lymphoma, prostate cancer, rectal cancer, renal pelvis and ureter cancer, retinoblastoma, rhabdomyosarcoma, salivary gland cancer, sarcoma (e.g. Ewing family, Kaposi, soft tissue, uterine), Sézary syndrome, skin cancer (e.g. nonmelanoma, Merkel cell), small cell lung cancer, small intestine cancer, soft tissue sarcoma, squamous cell carcinoma, squamous neck cancer, stomach cancer, supratentorial primitive neuroectodermal tumor, t-cell lymphoma, testicular cancer, throat cancer, thymoma and thymic carcinoma, thyroid cancer, trophoblastic tumors, urethral cancer, uterine cancer, uterine sarcoma, vaginal cancer, visual pathway and hypothalamic glioma, vulvar cancer, Waldenström macroglobulinemia, and Wilms tumor.

56. The transformed cell of claim 55, wherein the cancer is melanoma.

57. The transformed cell of claim 55, wherein the cancer is prostate cancer.

58. The transformed cell of claim 55, wherein the cancer is glioblastoma.

59. The transformed cell of claim 55, wherein the cancer is breast cancer.

60. The transformed cell of claim 55, wherein the cancer is ovarian cancer.

61. The transformed cell of claim 51, wherein the transformed cell is metastatic.

62. The transformed cell of claim 51, wherein the cell is a non-cancer cell.

63. A transformed cell, comprising: a variant or mutant PCBP1 polypeptide comprising one or more variations or mutations selected from the group consisting of: S223L, T60A, and T127A, relative to the wild type PCBP1 polypeptide of SEQ ID NO: 2, wherein the transformed cell is from a metastatic cancer.
